(19) United States
(12) Patent Application Publication
(10) Pub. No.: US 2006/0234317 A1
(43) Pub. Date: Oct. 19, 2006

(54) Method of screening

(76) Inventors: Rebecca Ada O'Donnell, Melbourne Victoria (AU); Tania Frances De Koning-Ward, Kilclare St. Burwood (AU); Brendan Scott Crabb, Melbourne (AU)

Correspondence Address: Leopold Presser;Scully Scott Murphy & Presser, 400 Garden City Plaza, Garden City, NY, 11530, US

(21) Appl. No.: 10/504,177

(22) PCT Filed: Feb. 11, 2003
(86) PCT No.: PCT/AU03/00142

§ 371 Date: May 31, 2005

Related U.S. Application Data
(60) Provisional application No. 60/355,897, filed on Feb. 11, 2002.

(51) Int. Cl. A61K 49/00; G01N 33/569; C12N 1/10

(52) U.S. Cl. 435/7.22; 435/258.2; 424/9.2

(57) ABSTRACT

A method of detecting the presence of a functionally inhibitory immunointeractive molecule in a biological sample. Preferably, the immunointeractive molecule is directed to a pathogen derived antigen and, more particularly, a parasite derived antigen and, even more particularly, a Plasmodium drived antigen. The method of the present invention facilitates detection of the presence of functionally inhibitory immunointeractive molecules, both in vitro and in vivo, and is useful for qualitatively and/or quantitatively assessing the immune status of individuals who have been previously infected with a parasite, predicting the immune status of individuals vaccinated with an antigen based vaccine, determining the relative contribution of a specific immunoreactivity of antibody to the total inhibitory antibody elicited by combination vaccines which include two or more antigens, assessing vaccines to determine the efficacy of different forms of an antigen, determining vaccine potency, assessing the protective potential of certain immunoreactivities of antibodies and determining the importance of parasite inhibitory antibodies.

### FIELD OF THE INVENTION

The present invention relates generally to a method of detecting the presence of an immunointeractive molecule in a biological sample. More particularly, the present invention relates to a method of detecting the presence of a functionally inhibitory immunointeractive molecule in a biological sample. Preferably, said immunointeractive molecule is directed to a pathogen derived antigen and, even more particularly, a parasite derived antigen. The method of the present invention facilitates detection of the presence of functionally inhibitory immunointeractive molecules, both in vitro and in vivo, and is useful, inter alia, for qualitatively and/or quantitatively assessing the immune status of individuals who have been previously infected with a parasite, predicting the immune status of individuals vaccinated with an antigen based vaccines, determining the relative contribution of a specific immunoreactivity of antibody to the total inhibitory antibody elicited by combination vaccines which include two or more antigens, assessing vaccines to determine the efficacy of different forms of an antigen, determining vaccine potency, assessing the protective potential of certain immunoreactivities of antibodies and determining the importance of parasite inhibitory antibodies.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Infection by the protozoan parasite Plasmodium falciparum results in several hundred million clinical cases of malaria each year of which approximately two million are fatal. the development of a malaria vaccine is now a major global initiative. Progress toward this goal requires an understanding of the mechanisms that underpin both naturally acquired and vaccine-induced immunity. Antibodies that inhibit the growth of bloodstage P. falciparum parasites in vitro are found in the sera of some, but not all, individuals living in malaria endemic regions (Marsh, K., Otoo, L., Hayes, R. J., Carson, D. C. and Greenwood, B. M. (1989) Trans. R. Soc. Trop. Med Hyg 83:293-303; Brown, G. V., Anders, R. F., Mitchell, G. F. and Heywood, P. F. (1982) Nature 297:591-593; Brown, G. V., Anders, R. F. and Knowles, G. (1983) Infect. Immun. 39:1228-1235; Bouharoun-Tayoun, H., Attanath, P., Sabchareon, A., Chongsuphajaisiddhi, T. and Druilhe, P. (1990) J. Exp. Med. 172:1633-1641). Inhibitory antibodies are likely to contribute to the clinical immunity observed in highly exposed individuals but their overall significance to protection remains unclear (Mohan, K. and Stevenson, M. M. (1998) Acquired immunity to asexual blood stages. In Malaria, parasite Biology, Pathogenesis and Protection. I. W. Sherman, editor. ASM Press, Washington, D.C. 467-493; McGregor, I. A. and Wilson, R. M. J. (1988) Specific immunity acquired in man. In Malaria, Principles and Practices of Malariology. W. H. Wernsdorfer and I. A. McGregor, editors. Churchill Livingston, Inc., New York. 559-619).

Inhibitory antibodies function by preventing invasion of red blood cells by the extracellular merozoite form of the parasite. A number of merozoite antigens have been shown to be targets of invasion inhibitory antibodies including some that localize to the merozoite surface, parasitophorous vacuole, and apical organelles. One target of inhibitory antibodies is the membrane-associated 19-kD COOH-terminal fragment of merozoite surface protein (MSP)′-1₁₉, a molecule that is now a leading malaria vaccine candidate (Digs, C. L., Ballou, W. R. and Miller, L. H. (1993) Parasitol. Today. 9:300-302; Good, M. F., Kaslow, D. C. and Miller, L. H. (1998) Annu. Rev. Immunol. 16:57-87). MSP-1₁₉ is unknown, however, allelic replacement experiments have shown that the function of most of the two EGF domains is conserved across distantly related Plasmodium species (O'Donnell, R. A., Saul, A., Cowman, A. F. and Crabb, B. S. (2000) Nat. Med. 6:91-95). The MSP-1₁₉ EGF domains form reduction-sensitive epitopes that are recognised by invasion-inhibitory monoclonal and polyclonal antibodies (O'Donnell, R. A. et al. 2000 supra; Blackman, M. J., Heidrich, H.-G., Donachie, S., McBridge, J. S. and Holder, A. A. (1990) J. Exp. Med 172:379-382; Chappel, J. A. and Holder, A. A. (1993) Mol. Biochem. Parasitol. 60:303-311; Cooper, J. A., Cooper, L. T. and Saul, A. J. (1992) Mol. Biochem. Parasitol. 51:301-312; Chang, S. P., Gibson, H. L., Lee Ng, C. T., Bar, P. J. and Hui, G. S. (1992) J. Immunol. 149:548-555). MSP-1₁₉-specific inhibitory antibodies are also present in the sera of individuals naturally exposed to P. falciparum (Egan, A., Burghaus, P., Druilhe, P., Holder, A. and Riley, E. (1999) Parasite Immunol. 21:133-139). These antibodies recognise epitopes formed by the double EGF domain and by the second EGF domain alone (Egan, A., Burghaus, P., Druilhe, P., Holder, A. and Riley, E. (1999) Parasite Immunol. 21:133-139). The mechanism of inhibition by MSP-1₁₉ antibodies is not fully understood, however, those that prevent the secondary processing of a precursor molecule and hence the formation of MSP-1₁₉ also effectively inhibit merozoite invasion of RBCs (Blackman, M. J., Scott Finnigan, T. J., Shai, S. and Holder, A. A. (1994) J. Exp. Med. 180:389-393).

In light of the extensive research and development which is now directed to the development of a malaria vaccine, it is clearly necessary that there are available rapid and accurate methods of qualitatively and/or quantitatively screening for the presence of an immune response to this parasite. To date, most such screening assays have been based on an analysis of the presence of antibody molecules based on binding of the antibody to the pathogen of interest. These results have been obtained using methods such as ELISA. However, such assays do not discriminate between the presence of immunointeractive molecules which bind but which do not further impact on the functional activity of the pathogen to which they bind versus immunointeractive molecules which do impact on this functioning. For example, it is known that some specificities of antibodies which are generated to the malaria MSP-1₁₉ antigen do not impact on the functional activity of the parasite, while others do. Such functionally inhibitory antibodies are particularly useful because in addition to facilitating the induction of various antibody related clearance mechanisms, they down-regulate the functional activity of the malaria parasite by, inter alia, inhibiting its ability to infect red blood cells. Clearly, where one is seeking to induce an immune response which either inhibits or clears a malaria infection, it is desirable to focus on the induction of an immune response which is inhibitory to the viability, functioning and/or proliferation of the parasite in addition to facilitating the induction of traditional clearance mechanisms. Further, to the extent that the generation of such an immune response is essential in order to achieve effective immunity, it is necessary that one has access to screening assays which can detect and measure the quality of an immune response which an individual has generated.

Accordingly, there is a need to develop more sophisticated screening assays which are able to analyse the immunointeractive molecule component of a biological sample at both the qualitative and quantitative levels, in particular, assays which are able to analyse the functional impact of an immunointeractive molecule on a given pathogen.

In work leading up to the present invention, the inventors have developed a means of detecting the presence of a functionally inhibitory immunointeractive molecule, in particular an antibody, as opposed to merely detecting the absolute levels of an immunointeractive molecule on the basis of binding specificity alone. This objective is achieved by analysing a functional pathogen parameter, such as pathogen growth for example, of a pathogen expressing the native form of the antigen of interest, which pathogen has been contacted with the purported immunointeractive molecule sample, relative to that of a pathogen which has been genetically altered such that it expresses an epitopically different form of the antigen in issue. Where inhibition of the subject functional parameter is observed in cultures of the pathogen expressing the native form of antigen relative to the genetically altered cultures, there is indicated the presence of functionally inhibitory antibody in the sample. However, where no difference is observed in the functional output of the native antigen cultures relative to the genetically altered cultures, there is indicated the absence of functionally inhibitory antibody (despite the fact that standard immunointeractivity based assays such as ELISAS may indicate the presence of immunointeractive molecules, such as antibodies, which are nevertheless binding to the pathogen in issue). The development of this correlate-of-protection assay now facilitates the analysis of the quality of an immune response. Further, these developments have facilitated the development of both in vitro and in vivo based screening methods.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides a method of detecting the presence of a functionally modulatory immunointeractive molecule in a biological sample, which immunointeractive molecule is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
   wherein modulation in the functional activity of the pathogen of step (ii) relative to the pathogen of step (i) is indicative of the presence of a functionally inhibitory immunointeractive molecule in said sample.

Another aspect of the present invention provides a method of detecting the presence of a functionally inhibitory immunointeractive molecule in a biological sample, which immunointeractive molecule is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
   wherein a decrease in the functional activity of the pathogen of step (ii) relative to the pathogen of step (i) is indicative of the presence of a functionally inhibitory immunointeractive molecule in said sample.

Yet another aspect of the present invention provides a method of detecting the presence of a functionally inhibitory antibody in a biological sample, which antibody is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
   wherein a decrease in the functional activity of the pathogen of step (ii) relative to the pathogen of step (i) is indicative of the presence of a functionally inhibitory antibody in said sample.

Still another aspect of the present invention provides a method of detecting the presence of a functionally inhibitory antibody in a biological sample, which antibody is directed to a parasite derived antigen, said method comprising:
(i) contacting a parasite expressing an epitopically distinct form of said antigen with said sample for a time and under conditions sufficient to facilitate immunointeraction;
(ii) contacting a parasite expressing an epitopically native form of said antigen with said sample for a time and under conditions sufficient to facilitate immunointeraction;
(iii) assessing the level of functional activity of the parasites of step (i) and step (ii)
   wherein a decrease in the functional activity of the parasite of step (ii) relative to the parasite of step (i) is indicative of the presence of a functionally inhibitory antibody in said sample.

In still yet another aspect there is provided a method of detecting the presence of a functionally inhibitory antibody in a biological sample, which antibody is directed to a Plasmodium derived antigen, said method comprising:
(i) contacting Plasmodium expressing an epitopically distinct form of said antigen with said sample for a time and under conditions sufficient to facilitate immuno interaction;
(ii) contacting Plasmodium expressing an epitopically native form of said antigen with said sample for a time and under conditions sufficient to facilitate immunointeraction;
(ii) assessing the level of inhibition of red blood cell invasiveness of Plasmodium of step (i) and step (ii)
   wherein a decrease in the red blood cell invasiveness of the Plasmodium of step (ii) relative to the Plasmodium of step (i) is indicative of the presence of a functionally inhibitory antibody in said sample.

In yet still another aspect there is provided a method of detecting the presence of a functionally inhibitory antibody in a biological sample, which antibody is directed to Plasmodium falciparum MSP-1, said method comprising:
(i) contacting Plasmodium falciparum expressing an epitopically distinct form of MSP-1 with said sample for a time and under conditions sufficient to facilitate immunointeraction;
(ii) contacting Plasmodium falciparum expressing an epitopically native form of MSP-1 with said sample for a time and under conditions sufficient to facilitate immunointeraction;
(iii) assessing the level of functional activity of the Plasmodia of step (i) and step (ii) wherein a decrease in the functional activity of the Plasmodium falciparum of step (ii) relative to the Plasmodium falciparum of step (i) is indicative of the presence of a functionally inhibitory antibody in said sample.

Preferably, said MSP-1 is the block 17 C-terminal domain or the block 2 N-terminal domain of MSP-1.

In a further aspect there is provided a method of detecting the presence of a functionally inhibitory antibody in a biological sample, which antibody is directed to Plasmodium falciparum AMA-1, said method comprising:
(i) contacting Plasmodium falciparum expressing an epitopically distinct form of AMA-1 with said sample for a time and under conditions sufficient to facilitate immunointeraction;
(ii) contacting Plasmodium falciparum expressing an epitopically native form of AMA-1 with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said AMA-1 is domain 3 of AMA-1.

In yet another further aspect there is provided a method of detecting the presence of a functionally inhibitory antibody in a biological sample, which antibody is directed to any one or more of Plasmodium falciparum MSP-2, MSP-3, MSP-4 and/or MSP-5, said method comprising:
(i) contacting Plasmodium falciparum expressing an epitopically distinct form of MSP-2, MSP-3, MSP-4 and/or MSP-5 with said sample for a time and under conditions sufficient to facilitate immunointeraction;
(ii) contacting Plasmodium falciparum expressing an epitopically native form of MSP-2, MSP-3, MSP-4 and/or MSP-5 with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

In still yet another further aspect there is provided a method of detecting the presence of a functionally inhibitory antibody in a biological sample, which antibody is directed to Plasmodium falciparum RAP-2 and/or RAP-1, said method comprising:
(i) contacting Plasmodium falciparum expressing an epitopically distinct form of RAP-2 and/or RAP-1 with said sample for a time and under conditions sufficient to facilitate immunointeraction;
(ii) contacting Plasmodium falciparum expressing an epitopically native form of RAP-2 and/or RAP-1 with said sample for a time and under conditions sufficient to facilitate immunointeraction;
(iii) assessing the level of functional activity of the Plasmodia of step (i) and step (ii) wherein a decrease in the functional activity of the Plasmodium falciparum of step (ii) relative to the Plasmodium falciparum of step (i) is indicative of the presence of a functionally inhibitory antibody in said sample.

Preferably, said RAP-1 is the N-terminal region of RAP-1.

In a further aspect there is provided a method of detecting the presence of a functionally inhibitory antibody in a biological sample, which antibody is directed to Plasmodium falciparum erythrocyte binding antigen, said method comprising:
(i) contacting Plasmodium falciparum expressing an epitopically distinct form of erythrocyte binding antigen with said sample for a time and under conditions sufficient to facilitate immunointeraction;
(ii) contacting Plasmodium falciparum expressing an epitopically native form of erythrocyte binding antigen with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said erythrocyte binding antigen is EBA-175 and even more preferably the F2 domain of EBA-175.

In another further aspect there is provided a method of detecting the presence of a functionally inhibitory antibody in a biological sample, which antibody is directed to Plasmodium falciparum CSP, said method comprising:
(i) contacting Plasmodium falciparum expressing an epitopically distinct form of CSP with said sample for a time and under conditions sufficient to facilitate immunointeraction;
(ii) contacting Plasmodium falciparum expressing an epitopically native form of CSP with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said CSP-1 is the block 2 N-terminal domain of CSP-1.

Another aspect of the present invention provides a method of detecting the presence of a functionally inhibitory antibody in a biological sample, which antibody is directed to Plasmodium falciparum MSP-1₁₉, said method comprising:
(i) contacting a Plasmodium falciparum schizont of strain D10-PcM3′ with said sample for a time and under conditions sufficient to facilitate immunointeraction;
(ii) contacting a Plasmodium falciparum schizont of the strain D10 with said sample for a time and under conditions sufficient to facilitate immunointeraction;
(iii) assessing the level of functional activity of the Plasmodium falciparum strains of step (i) and step (ii);
   wherein a decrease in the functional activity of the Plasmodium falciparum strain of step (ii) relative to the Plasmodium falciparum strain of step (i) is indicative of the presence of a functionally inhibitory antibody in said sample.

In another aspect there is provided a method of detecting the presence of a functionally inhibitory antibody in a biological sample, which antibody is directed to Plasmodium falciparum MSP-1₁₉, said method comprising:
(i) contacting a Plasmodium falciparum schizont of strain D10 with said sample for a time and under conditions sufficient to facilitate immunointeraction;
(ii) contacting a Plasmodium falciparum schizont of strain D10-PcMEGF with said sample for a time and under conditions sufficient to facilitate immunointeraction;
(iii) assessing the level of functional activity of the Plasmodium falciparum strains of step (i) and step (ii);
   wherein a decrease in the functional activity of the Plasmodium falciparum strain of step (ii) relative to the Plasmodium falciparum strain of step (i) is indicative of the presence of a functionally inhibitory antibody in said sample.

Accordingly, in yet another embodiment there is provided a method of detecting the presence of a functionally inhibitory antibody in a population of mice, which antibody is directed to Plasmodium falciparum MSP-1 and which method is performed in vivo in said mice, said method comprising:
(i) introducing to at least one of said mice a wild-type Plasmodium berghei for a time and under conditions sufficient to facilitate immunointeraction;
(ii) introducing to at least one of said mice, other than the mouse of step (i), a Plasmodium berghei strain, which strain expresses the Plasmodium falciparum MSP-1 block 17C-terminal domain, for a time and under conditions sufficient to facilitate immunointeraction;
(iii) assessing the level of functional activity of the Plasmodia of step (i) and step (ii)
   wherein a decrease in the functional activity of the Plasmodium berghei of step (ii) relative to the Plasmodium berghei of step (i) is indicative of the presence of said functionally inhibitory antibody in said mice.

Most preferably, there is provided a method of detecting the presence of a functionally inhibitory antibody in a population of mice, which antibody is directed to Plasmodium falciparum MSP-1₁₉ and which method is performed in vivo in said mice, said method comprising:
(i) introducing to at least one of said mice a wild-type Plasmodium berghei for a time and under conditions sufficient to facilitate immunointeraction;
(ii) introducing to at least one of said mice, other than the mouse of step (i), a Plasmodium berghei schizont of the strain Pb-PfM19 for a time and under conditions sufficient to facilitate immunointeraction;
(iii) assessing the level of functional activity of the Plasmodium berghei strains of step (i) and step (ii)
   wherein a decrease in the functional activity of the Plasmodium berghei strain of step (ii) relative to the Plasmodium berghei strain of step (i) is indicative of the presence of said functionally inhibitory antibody in said mouse.

In still another aspect the present invention is directed to a method of assessing the nature of an immune response to an antigen in accordance with the methods defined hereinbefore.

In yet another aspect, the present invention extends to the pathogens defined herein.

Accordingly, yet another aspect of the present invention is directed to an isolated pathogen, which pathogen expresses a non-wild-type form of one or more antigens derived from said pathogen.

More particularly, the present invention provides an isolated malaria pathogen, which pathogen expresses a non-wild-type form of one or more antigens derived from said pathogen.

Preferably, the present invention provides an isolated Plasmodium, which Plasmodium expresses a non-wild-type form of MSP-1.

More preferably, said MSP-1 is the block 17 C-terminal domain or the block 2 N-terminal domain of MSP-1.

Most preferably, said Plasmodium is Plasmodium berghei expressing the Plasmodium falciparum form of the MSP-1₁₉ antigen.

Even more preferably, said Plasmodium berghei is the Pb-PfM19 strain.

In another embodiment, the present invention provides an isolated Plasmodium pathogen expressing a non-wild-type form of one or more antigens derived from said pathogen, which antigens are selected from the list of:
(i) the apical membrane domain (AMA-1)
(ii) merozoite surface protein 2, 3, 4 and/or 5 (MSP-2, MSP-3, MSP-4 and/or MSP-5)
(iii) rhoptry associated protein 2 (RAP-2)
(iv) erythrocyte binding antigens (EBA-175)
(v) circumsprozoite antigen (CSP)

In yet another aspect, the present invention extends to the pathogens defined herein when used in accordance with the method of the present invention.

### BRIEF DESCRIPTION OF THE DRAWINGS

FIG. 1 is An image showing generation of a transfected P. falciparum line containing the complete MSP-1₁₉ EGF domains from P. chabaudi in place of the endogenous molecule. (A) Alignment of MSP-1₁₉ sequences from P. falciparum (MAD20 allele; GenBank/EMBL/DDBJ accession no. M19143) and P. chabaudi (adami DS line; GenBank/EMBL/DDBJ accession no. AF149303). The arrows indicate the sites of secondary cleavage, asterisks denote identical residues, and dots highlight conserved residues. The disulfide bonds expected for EGF-like domains are shown (black lines). Note the absent disulfide bond in P. chabaudi (dashed line). The nature of the gene fusions in the various MSP-1 hybrid lines is represented underneath the alignment with the dashed line representing endogenous P. falciparum sequence and the solid line, P. chabaudi sequence. (B) The plasmid pPcMEGF was constructed by ligating a DNA fragment containing P. falciparum MSP-1 sequence (Target) fused to sequence encoding MSP-1₁₉ from P. chabaudi MSP-1 (dark shading) into the XhoI site of pHC2. The predicted structure of the MSP-1 loci following integration of pPcMEGF and the location of the XbaI (X) sites used to map these loci are shown. The location of XbaI sites unique to D10-PcM3′ and D10-PfM3′ are bracketed and are represented as X.Pc and X.Pf, respectively. All sizes are to scale with the exception of the plasmid backbone (dashed line). (C) Southern blot analysis of gDNA restricted with XbaI showing that pPcMEGF had integrated into MSP-1 as predicted and that the resultant line (D10-PcMEGF) differs from the previously established lines D10-PfM3′ and D10-PcM3′ (O'Donnell, R. A. et al., 2000). The 0.9-kb PfMSP-1 fragment (Target) was used to probe the blot.

FIG. 2 is an image of transfected D10-PcMEGF parasites express a functional MSP-1 chimera. (A) Western blot analysis of parasite proteins from extracted enriched schizont (Schiz) or merozoite (Mer) preparations of parental D10 and the D10-PcMEGF clones (PcMEGF.1 and PcMEGF.2). Proteins were separated by SDS-PAGE under non-reducing conditions, transferred to PVDF membranes, and probed with either 4H9/19 or *PcM19 antibodies as indicated. The position of molecular weight standards are shown to the left and are in Kd. (B) Localization of MSP-1 expressed in the transfected lines by indirect IFA. D10-PfM3′ (PfM3′) and D10-PcMGF.1 (PcMEGF.1) schizont-stage parasites were incubated with a mixture of 4H9/19 and *PcM19 antibodies. After incubation in the presence of a mixture of FITC-conjugated anti-mouse and rhodamine-conjugated anti-rabbit Igs, parasites were visualised by microscopy. Original magnification: 1,000×. the same fields were photographed under fluorescence conditions to detect the FITC or rhodamine fluorochromes. (C) In vitro inhibition assays of D10, D10-PcM3′, and D10-PcMEGF clones (PcMEGF.1 and PcMEGF.2) with different concentrations of *PcM19 antibodies (IgG). Error bars represent SDs.

FIG. 3 is a graphical representation of the invasion inhibition of transfected P. falciparum parasites expressing divergent MSP-1₁₉ domains by sera from clinically immune individuals reveals an important role for MSP-1₁₉-specific antibodies. (A) Assay 1, microscopy. Microscopy-based invasion inhibition assay involving the detection of ring-stage D10 and D10-PcMEGF (PCMEGF) parasites after cultivation in the presence of each individual serum. (B) Assay 2, hypoxanthine uptake. Alternative, invasion-inhibition assay comparing D10-PfM3′ (PfM3′) and D10-PcMEGF parasites using [³H]hypoxanthine uptake as a measure of parasite growth. Invasion is represented as either parasiternia (A) or counts (B) and is expressed as a percentage of the invasion observed in parasites cultured in negative control sera (HNIS). The means of samples within a serum set against each parasite line are indicated. P values from a Student's t test comparing the means in each panel are shown.

FIG. 4 is a graphical representation of the invasion-inhibition assay with representative individual sera from PNG-B and Pc-immune serum sets against D10-PfM3′ and D10-PcMEGF parasite lines. Samples were selected from assay 2 and represent typical examples of the inhibitory activities observed. The results obtained for the control sera in assay 2, anti-P. falciparum AMA-1 (*PfAMA1), and *PcM19 IgG are shown. Error bars represent the range observed in duplicate samples.

FIG. 5 is an image of co-cultivation of D10-PfM3′ and D10-PcMEGF parasites in the presence of immune sera confirms an important role for MSP-1₁₉ antibodies in invasion inhibition. Ring-stage D10-PfM3′ and D10-PcMEGF parasites were combined at an equal ratio and cultured in the presence of the pooled sera indicated at right. Smears from days 1 and 5 were analyzed by double-labelling IFA. Mature stage (pigmented) green and red parasites were counted in 16 fields each containing at least 10 parasites. The same fields were observed by fluorescence microscopy using filters to detect the FITC or rhodamine fluorochromes. Results are expressed as a ratio of D10-PfM3′ to D10-PcMEGF. A representative field of parasites (at day 3) cultured in the presence of HNIS pool is shown (inset).

FIG. 6 is an image of the functional complementation of divergent MSP-1₁₉ domains in vivo: Replacement of the P. berghei MSP-119 domain with that from P. falciparum MSP-1₁₉ (MAD20 allele) in P. berghei parasites cultured in mice. (A) Schematic diagram showing the P. berghei MSP-1 locus before (top) and after (bottom) homologous integration of the pPb-PfM19 transfection plasmid. Within the plasmid, the location of the 5′ and 3′ homologous sequences used for gene targeting (solid lines), the P. falciparum MSP-1₁₉ sequence (black box), the HSP863′ region (3′) and the selectable marker (Tg DHFR-TS cassette) are shown. The location of HincII (H), EcoRI (E) and SwaI (S) restriction sites are shown. (B) Southern blot of HincII (left) or EcoRI/SwaI (right) digested genomic DNA from wild type P. berghei (Pb WT) or transfected P. berghei (Pb-PfM19). The location of the probe is shown (solid dashed line). The presence of bands of the expected sizes and the absence of an endogenous wil type band in the Pb-PfM19 lanes is indicative of a pure population of transfected possessing the expected double-crossover homologous integration event.

This data demonstrates that rodent malaria parasites (P. berghei) are viable in mice when expressing a MSP-1 hybrid molecule incorporating the complete MSP-1₁₉ domain from P. falciparum in place of endogenous P. berghei MSP-1. This is the first demonstration that these domains are functionally conserved across divergent Plasmodium species in in vivo cultured blood-stage parasites.

FIG. 7 is a schematic representation of P. berghei and P. falciparum MSP-1 chimeras. The MSP-1 sequences of P. berghei (grey), P. falciparum (red) and P. chabaudi (blue) are represented. The Pb-PbM19 control chimera (this study) is identical at the MSP-1 locus to wildtype P. berghei, whereas the Pb-PfM19 chimera (this study) expresses P. falciparum MSP-1₁₉ in place of the endogenous molecule. Likewise, D10-PfM3′ (21), is identical at the MSP-1 locus to wildtype P. falciparum, while D10-PcMEGF expresses the P. chabaudi MSP-1₁₉ polypeptide (9). The arrows indicate the MSP-1 secondary cleavage site.

FIG. 8 is a schematic representation of the generation of P. berghei chimera lines containing either P. berghei or P. falciparum MSP-1₁₉. (A) Schematic diagram of the P. berghei MSP-1 locus, the transfection vector (pPb-PfM19) used to replace the endogenous MSP-1₁₉ molecule, and the predicted MSP-1 locus of the Pb-PfM19 chimeric line after integration. The grey box represents endogenous P. berghei MSP-1₉ sequence while the black box represents P. falciparum MSP-1₁₉ sequence. The solid lines in pPb-PfM19 depict targeting sequence used to drive integration. The same strategy was used to create the Pb-PbM19 chimeric line, with the exception that the sequence represented by the black box is that of P. berghei MSP-1₁₉. Tg DHFR-TS, selectable marker cassette; 3′, HSP86 3′ UTR. The expected sizes of fragments resulting from digestion with either HincII (H) or PstI (P) are shown. (B) Southern blot analysis of digested genomic DNA from P. berghei wiltype and chimeric lines. Replacement of the endogenous MSP-1₁₉ sequence with that of P. falciparum (Pb-PfM19 chimera) or wiltype P. berghei sequence (Pb-PbM19 chimera) was confirmed by the hybridisation of Southern blots with either probe A or B as shown in FIG. 2A.

FIG. 9 is an image of the phenotypic analysis of P. berghei chimeric lines. (A) Western blot analysis of late stage parasite extracts using rabbit αPbM19 or αPfM19 antibodies (both diluted 1/4000) demonstrates that both full-length MSP-1 (approximately 200 kDa) and MSP-1₁₉ (approximately 19 kDa) could be detected in wildtype and chimeric P. berghei lines. (B) Localisation of MSP-1₁₉ in wildtype and chimeric P. berghei lines by indirect immunofluorescence assay. Schizont-stage parasites were incubated with a mixture of αPbM19 ( 1/1000) and 4H9/19 ( 1/100) antibodies, followed by a mixture of FITC-conjugated anti-rabbit and rhodamine-conjugated anti-mouse immunoglobulins (both diluted 1/200). The same fields were photographed under fluorescence conditions to detect the FITC or rhodamine fluorochromes. (C) Course of blood parasitemia in mice following infection at Day 0 with P. berghei wildtype, Pb-PbM19 or Pb-PfM19. Shown is the mean±SD of the parasitemia observed in 5 mice.

FIG. 10 is a graphical representation of mice repeatedly infected with P. berghei transfectants eliciting MSP-1₁₉ specific inhibitory antibodies. (A) Anti-MSP-1₁₉ antibody endpoint titres of serum from Pb-PfM19 and Pb-PbM19 immune mice against recombinant P. falciparum and P. berghei MSP-1₁₉—GST fusion proteins. (B) Invasion inhibition assay of D10-PfM3′ and D10-PcMEGF parasite lines in the presence of individual serum from Pb-PfM19 and Pb-PbM19 immune mice. The invasion rate is expressed as a percentage of the invasion observed in parasites cultured in human non-immune sera (HNIS). The numbers shown represent the P. falciparum MSP-1₁₉ specific invasion inhibitory activity of a given serum, calculated by subtracting the invasion rate of D10-PfM3′ from that of D10-PcMEGF.

FIG. 11 is a graphical representation of the evidence that MSP-1₁₉ specific inhibitory antibodies control a blood-stage infection. MSP-1₁₉ specific invasion inhibitory activity of serum from individual Pb-PfM19 immune mice plotted against the log of the peak parasitemia attained after challenging corresponding mice with Pb-PfM19.

### DETAILED DESCRIPTION OF THE INVENTION

The present invention is predicated, in part, on the determination that functionally inhibitory immunointeractive molecules, such as antibodies, can be both qualitatively and quantitatively identified where one measures a functional parameter of the pathogen of interest, which functional parameter is that which would be down-regulated and/or inhibited in the presence of the immunointeractive molecule of interest, and where one analyses this parameter relatively to that of a genetically altered pathogen which expresses an epitopically distinct form of the antigen which is the target of the immunointeractive molecule of interest. This determination has now facilitated the development of in vitro and in vivo assays directed to screening for immunointeractive molecules (in particular, antibodies) which, in addition to binding to a pathogen, also act to inhibit or otherwise down-regulate one or more of the pathogen's functional attributes. Although the method of the present invention is exemplified with respect to malaria, it can be applied to any pathogen and now facilitates the analysis of the functional quality of an immune response, which type of analysis was not previously available. The method of the present invention is applicable in a range of situations including, but not limited to, the assessment of the quality of an individual's immunity, the determination of whether a vaccine protocol is inducing a functionally relevant form of immunity or to determining the relative contribution of a specific immunointeractive molecule to the total inhibitory functioning of a given immune response.

Accordingly, one aspect of the present invention provides a method of detecting the presence of a functionally modulatory immunointeractive molecule in a biological sample, which immunointeractive molecule is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
   wherein modulation in the functional activity of the pathogen of step (ii) relative to the pathogen of step (i) is indicative of the presence of a functionally inhibitory immunointeractive molecule in said sample.

Reference to “modulation” should be understood as a reference to up-regulation or down-regulation. Although the preferred method is to detect immunointeractive molecules which down-regulate functional activity, there may be circumstances in which it is desirable or necessary to screen for molecules which aberrantly, or otherwise, act to up-regulate the functional activity of a pathogen.

Accordingly, the present invention more particularly provides a method of detecting the presence of a functionally inhibitory immunointeractive molecule in a biological sample, which immunointeractive molecule is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
   wherein a decrease in the functional activity of the pathogen of step (ii) relative to the pathogen of step (i) is indicative of the presence of a functionally inhibitory immunointeractive molecule in said sample.

The inventors in respect of the present invention have actually developed two aspects in respect of this method of screening, these being its application in an in vitro environment and its application in an in vivo environment. The development of an in vivo assay, in particular, is a surprising and unusual development which now facilitates forms of analysis which were not previously available, and which would not be available even utilising the in vitro methodology herein described.

In relation to the in vivo methodology which is disclosed herein, the inventors have specifically developed a non-human animal model method for detecting, in vivo, functionally inhibitory antibodies. This method is based on the determination that the infection of a non-human animal with a pathogen expressing an antigen of interest, which pathogen is one which is both suitable for colonising the selected animal model and the activity of which will be modulated if bound by a functionally inhibitory antibody directed to said antigen, provides a means for determining whether a biological sample which is introduced to said animal comprises functionally inhibitory molecules. This determination is based on a relative analysis of the functionality of pathogens expressing the native form of the antigen of interest versus those expressing an epitopically distinct form of said antigen.

It should be understood that such an in vivo detection method has extensive application. For example, in one embodiment, a murine model is infected with a murine malaria parasite expressing a form of the epitope of interest which is expressed by a human malaria parasite. There is thereby provided a means of screening a biological sample for the generation and/or presence of functionally inhibitory antibodies, directed to the human form of the epitope, which antibodies do not bind to the epitopically distinct murine homologue of the epitope. The person of skill in the art would recognise that the availability of such an in vivo screening model provides advantages which are not provided by an in vitro based screening assay, such as the ability to immunise the animal model with developmental vaccines which are designed to elicit an immune response to the epitope of interest. In this way, vaccines intended for use in humans can be trialed in a non-human model which will provide accurate results in respect of the quality of the immune response which is generated to that vaccine, in terms of its functional impact of the epitope of interest.

As would be appreciated by the person of skill in the art, the development of the in vitro assay herein described herein also provides unique advantages, such as the capacity to rapidly perform high throughput analysis.

Reference to “immunointeractive molecule” should be understood as a reference to any molecule which comprises an antigen binding portion. By “antigen” is meant any molecule against which an immune response may be generated. In accordance with the method of the present invention, the antigen is a pathogen. It should be understood that the subject immunointeractive molecule may take any form. For example, it may be a secreted form of a molecule, such as an antibody, or it may be linked, bound or otherwise associated with any other molecule, such as a cell. For example, a T cell receptor is likely to be associated with a T helper cell or a T cytotoxic cell. It should be understood that the molecule or cell may also be coupled to any other proteinaceous or non-proteinaceous molecule, such as a tag which facilitates its detection or tracking. The immunointeractive molecule may be naturally occurring or it may have been genetically or otherwise modified. Examples of molecules contemplated by this aspect of the present invention include, but are not limited to, monoclonal and polyclonal antibodies (including synthetic antibodies), hybrid antibodies, humanised antibodies, catalytic antibodies and T cell antigen binding molecules. Preferably, said immunointeractive molecule is an antibody. Reference to “antibody” hereinafter is not intended to be limiting and should be understood to include reference to any form of immunointeractive molecule.

The method of the present invention therefore still more particularly provides a method of detecting the presence of a functionally inhibitory antibody in a biological sample, which antibody is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
   wherein a decrease in the functional activity of the pathogen of step (ii) relative to the pathogen of step (i) is indicative of the presence of a functionally inhibitory antibody in said sample.

Reference to “pathogen” should be understood as a reference to any microorganism which can infect a human or non-human animal or to a molecule secreted therefrom. The subject pathogen may or may not result in the onset of a disease condition. In this regard, many pathogens do induce diseases. However, some pathogens can colonise an animal and exist in a symbiotic relationship without the onset of a disease condition. Such pathogens, due to their foreign nature, may nevertheless result in the onset of an acute or chronic immune response, the analysis of which response in accordance with the methods defined herein may be nevertheless desirable. Reference to “pathogen” should also be understood to encompass pathogens which have either naturally or non-naturally undergone some form of mutation, genetic manipulation or any other form of manipulation. Accordingly, the chimaeric Plasmodium falciparum strains disclosed herein should be understood to fall within the scope of the definition of “pathogen”. Examples of pathogens include, but are not limited to, bacteria, viruses and parasites. Preferably, the subject pathogen is a parasite and even more preferably a malaria inducing parasite.

The human or non-human animal as described herein includes humans, primates, livestock animals (eg. sheep, pigs, cows, horses, donkeys), laboratory test animals (eg. mice, rats, rabbits, guinea pigs), companion animals (eg. dogs, cats), captive wild animals (eg. foxes, kangaroos, deer), aves (eg. chicken, geese, ducks, emus, ostriches), reptiles or fish. Preferably, the subject is a human.

The present invention therefore more preferably provides a method of detecting the presence of a functionally inhibitory antibody in a biological sample, which antibody is directed to a parasite derived antigen, said method comprising:
(i) contacting a parasite expressing an epitopically distinct form of said antigen with said sample for a time and under conditions sufficient to facilitate immunointeraction;
(ii) contacting a parasite expressing an epitopically native form of said antigen with said sample for a time and under conditions sufficient to facilitate immunointeraction;
(iii) assessing the level of functional activity of the parasites of step (i) and step (ii)
   wherein a decrease in the functional activity of the parasite of step (ii) relative to the parasite of step (i) is indicative of the presence of a functionally inhibitory antibody in said sample.

Preferably, the subject parasite is a malaria inducing parasite.

“Malaria” is a term used to describe a class of diseases which are caused by infection with the protozoans of the genus Plasmodium. These diseases are also known by other names including Ague, Marsh Fever, Periodic Fever and Paludism. The Plasmodium species P. falciparum, P. malariae, P. ovale and P. vivax will each result in the onset of malaria in the human. In general, and without limiting the present invention in any way, the disease is transmitted by the Anopheles mosquito and is confined mainly to tropical and sub-tropical areas. Parasites in the blood of an infected person are taken up into the stomach of the mosquito as it feeds. Here, they multiply and then invade the mosquito salivary glands. When the mosquito bites a subject, parasites are simultaneously injected into the blood stream and thereafter migrate to the liver and other organs, where they multiply. After an incubation period varying from 12 days (P. falciparum) to 10 months (some varieties of P. vivax), parasites return to the blood stream and invade the red blood cells. Rapid multiplication of the parasites results in destruction of the red cells and the release of more parasites capable of infecting other red cells. This causes a short bout of shivering, fever and sweating and the loss of healthy red cells results in anaemia. When the next batch of parasites is released, symptoms reappear. The interval between fever attacks varies in different forms of malaria. For example, in Quartan malaria the interval is approximately three days and is caused by the species P. malaria. In Tertian malaria, the interval is two days and is caused by the species P. ovale and P. vivax. In malignant Tertian malaria, this being the most severe form of malaria, the interval is from a few hours to two days. This form of malaria is caused by P. falciparum.

Still without limiting the present invention in any way, the primitive malarial parasites which are injected by the mosquito are termed sporozoites. These sporozoites circulate in the blood for a short time and then settle in the liver where they enter the parenchymal cells and multiply. This stage is known as the pre-erythrocytic schizogony. After multiplication, there may be thousands of young parasites known as merozoites in one liver cell. At this time, the liver cell ruptures and the free merozoites enter red blood cells. In the red blood cells, the parasites develop into two forms, a sexual and an asexual cycle. The sexual cycle produces male and female gametocytes which circulate in the blood and are taken up by a female mosquito when taking a blood meal. In the asexual cycle, the developing parasites form schizonts in the red blood cells which contain many merozoites. The infected red cells rupture and release a batch of young merozoites which invade new red cells. The species P. vivax, P. ovale and P. malariae develop in the peripheral blood subsequently to the liver cycles. However, in the case of P. falciparum only ring forms and gametocytes are present in the peripheral blood.

Accordingly, it should be understood that many pathogens, in particular, parasites, pass through a number of developmental stages during their life cycle. Reference to “pathogen” in the context of the present invention and in particular in the context of steps (i) and (ii) as defined herein, should therefore be understood as a reference to a pathogen at any one of its life cycle developmental stage, whether that be a mature or immature developmental stage. For example, in the context of the embodiment exemplified herein, being the screening for P. falciparum MSP-1₁₉-directed functionally inhibitory antibodies, the P. falciparum pathogen which is utilised in steps (i) and (ii) may be of any suitable developmental stage. However, in accordance with the specific exemplification provided herein, ring stage parasites are synchronised and then allowed to mature through to the trophozoite/schizont stages prior to culturing, in accordance with steps (i) and (ii), with the biological sample of interest. It should be understood, however, that although this is a preferred form of conducting the subject screening test, the person of skill in the art may seek to use parasites at any other developmental stage, depending on the particular nature of the antigen against which immunointeractive antibodies are to be detected.

The method of the present invention is directed to screening for functionally inhibitory immunointeractive molecules, in particular functionally inhibitory antibodies. By “functionally inhibitory” is meant that the subject antibody, by virtue of binding, interacting or otherwise associating with a pathogen, acts to inhibit, prevent or otherwise down-regulate any one or more functional activities of that pathogen such as, but not limited to, division, maturation or cellular invasiveness. That is, the subject functional activity is inhibited by virtue of the association of the pathogen with a functionally inhibitory antibody, per se, and not necessarily by virtue of any subsequent clearance mechanism which may also occur (although such a possibility is not excluded by the present invention). For example, and without limiting the invention in any way, binding of certain antibody specificities to MSP-1₁₉ have been shown to prevent P. falciparum merozoites from further dividing and/or colonising red blood cells. It is not fully understood how the antibody achieves this outcome. Nevertheless, such antibodies are clearly highly desirable in an immune response since the traditional antibody based clearance mechanisms are not always effective in clearing or even controlling, certain types of parasitic infections.

In this regard, reference to assessing the level of “functional activity” of the pathogen should be understood as a reference to assessing the activity of the pathogen which corresponds to the activity which the functionally inhibitory antibody in issue would down-regulate. Although the present invention is exemplified in terms of screening for the modulation of a single functional activity, it should be understood that the person of skill in the art may screen for any one or more functional activities, for example, either because the person of skill in the art is simultaneously screening for the presence of a combination of functionally inhibitory antibodies or because the subject functionally inhibitory antibodies down-regulate more than one functional activity of the target pathogen. In accordance with the exemplification provided herein, in one embodiment the functionally inhibitory antibodies of interest is one which down-regulates the red blood cell invasiveness of Plasmodium falciparum merozoites. Accordingly, the functional activity which is the subject of screening is the capacity of P. falciparum merozoites, which have been cultured together with a test serum source to invade red blood cells. Nevertheless, it should be understood that a functionally inhibitory antibody, as defined herein, may be additionally involved in traditionally understood immune clearance mechanisms. However, it is its activity as an inhibitor of one or more pathogen functional activities which forms the basis of the detection of these antibodies in accordance with the method of the present invention. To the extent that the pathogen of interest is Plasmodium, the subject functional inhibition is preferably inhibition of red blood cell invasiveness.

Accordingly, there is provided a method of detecting the presence of a functionally inhibitory antibody in a biological sample, which antibody is directed to a Plasmodium derived antigen, said method comprising:
(i) contacting Plasmodium expressing an epitopically distinct form of said antigen with said sample for a time and under conditions sufficient to facilitate immunointeraction;
(ii) contacting Plasmodium expressing an epitopically native form of said antigen with said sample for a time and under conditions sufficient to facilitate immunointeraction;
(ii) assessing the level of inhibition of red blood cell invasiveness of Plasmodium of step (i) and step (ii)
   wherein a decrease in the red blood cell invasiveness of the Plasmodium of step (ii) relative to the Plasmodium of step (i) is indicative of the presence of a functionally inhibitory antibody in said sample.

Preferably, said Plasmodium is Plasmodium falciparum.

As detailed hereinbefore, the method of the present invention detects the presence of a functionally inhibitory antibody based on an analysis of the functional activity of a pathogen which has been contacted with a biological sample of interest. In this regard, reference to “pathogen derived antigen” should be understood as a reference to the antigen to which the subject functionally inhibitory antibody is directed. It should be understood that this antigen may form part of the pathogen itself or it may be a molecule which is secreted from the pathogen, the interaction of which with a functionally inhibitory antibody, for example, nevertheless acts to down-regulate one or more aspects of the functional activity of the pathogen itself or of that particular molecule. The subject antigen may be one which is either permanently or transiently expressed by the subject pathogen. The notion of transient expression of an antigen is likely to be of particular relevance with a pathogen such as a virus or parasite which passes through a number of distinct developmental life cycle stages.

It should be further understood that the subject antigen may comprise one or more epitopes, any one or more of which epitopes may be recognised by the antibody of interest. Alternatively, the subject antigen may be a very small antigen and may, in its entirety, correspond to a single epitope. In general, any given antibody of interest would only recognise one epitope of the antigen in issue, although cross-reactivity is nevertheless contemplated by the method of the present invention. The notion of an antibody expressing reactivity towards a single epitope accords with accepted immunological principles in relation to the specificity of antibody responses. In this regard, reference to the functionally inhibitory antibody being “directed” to the antigen should be understood to mean that the antibody recognises an epitope which is present on the antigen. To the extent that said pathogen is Plasmodium falciparum, said antigen is preferably any domain of MSP-1 (for example the “block 2” N-terminal domain or the block 17 C-terminal domain), the apical membrane domain (AMA-1), merozoite surface protein 2, 3, 4 and 5 (MSP-2, MSP-3, MSP-4 and MSP-5), rhoptry associated protein 2 (RAP-2), RAP-1, erythrocyte binding antigens (EBA-175) or the circumsprozoite antigen (CSP).

Accordingly, in one preferred embodiment there is provided a method of detecting the presence of a functionally inhibitory antibody in a biological sample, which antibody is directed to Plasmodium falciparum MSP-1, said method comprising:
(i) contacting Plasmodium falciparum expressing an epitopically distinct form of MSP-1 with said sample for a time and under conditions sufficient to facilitate immunointeraction;
(ii) contacting Plasmodium falciparum expressing an epitopically native form of MSP-1 with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said MSP-1 is the block 17 C-terminal domain or the block 2 N-terminal domain of MSP-1.

In another preferred embodiment there is provided a method of detecting the presence of a functionally inhibitory antibody in a biological sample, which antibody is directed to Plasmodium falciparum AMA-1, said method comprising:
(i) contacting Plasmodium falciparum expressing an epitopically distinct form of AMA-1 with said sample for a time and under conditions sufficient to facilitate immunointeraction;
(ii) contacting Plasmodium falciparum expressing an epitopically native form of AMA-1 with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said AMA-1 is domain 3 of AMA-1.

In yet another preferred embodiment, there is provided a method of detecting the presence of a functionally inhibitory antibody in a biological sample, which antibody is directed to any one or more of Plasmodium falciparum MSP-2, MSP-3, MSP-4 and/or MSP-5, said method comprising:
(i) contacting Plasmodium falciparum expressing an epitopically distinct form of MSP-2, MSP-3, MSP-4 and/or MSP-5 with said sample for a time and under conditions sufficient to facilitate immunointeraction;
(ii) contacting Plasmodium falciparum expressing an epitopically native form of MSP-2, MSP-3, MSP-4 and/or MSP-5 with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

In still yet another preferred embodiment, there is provided a method of detecting the presence of a functionally inhibitory antibody in a biological sample, which antibody is directed to Plasmodium falciparum RAP-2 and/or RAP-1, said method comprising:
(i) contacting Plasmodium falciparum expressing an epitopically distinct form of RAP-2 and/or RAP-1 with said sample for a time and under conditions sufficient to facilitate immunointeraction;
(ii) contacting Plasmodium falciparum expressing an epitopically native form of RAP-2 and/or RAP-1 with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said RAP-1 is the N-terminal region of RAP-1.

In a further preferred embodiment, there is provided a method of detecting the presence of a functionally inhibitory antibody in a biological sample, which antibody is directed to Plasmodium falciparum erythrocyte binding antigen, said method comprising:
(i) contacting Plasmodium falciparum expressing an epitopically distinct form of erythrocyte binding antigen with said sample for a time and under conditions sufficient to facilitate immunointeraction;
(ii) contacting Plasmodium falciparum expressing an epitopically native form of erythrocyte binding antigen with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

Preferably, said erythrocyte binding antigen is EBA-175 and even more preferably the F2 domain of EBA-175.

In another further preferred embodiment, there is provided a method of detecting the presence of a functionally inhibitory antibody in a biological sample, which antibody is directed to Plasmodium falciparum CSP, said method comprising:
(i) contacting Plasmodium falciparum expressing an epitopically distinct form of CSP with said sample for a time and under conditions sufficient to facilitate immunointeraction;
(ii) contacting Plasmodium falciparum expressing an epitopically native form of CSP with said sample for a time and under conditions sufficient to facilitate immunointeraction;
(iii) assessing the level of functional activity of the Plasmodia of step (i) and step (ii)
   wherein a decrease in the functional activity of the Plasmodium falciparum of step (ii) relative to the Plasmodium falciparum of step (i) is indicative of the presence of a functionally inhibitory antibody in said sample.

The method of the present invention overcomes previous shortcomings of antibody based screening methods wherein only the presence or absence of an antibody expressing a particular antigenic immunoreactivity could be measured. These methods (for example ELISAs, FACS analysis or immunofluorescent microscopy) cannot distinguish between those antibodies of a particular immunoreactivity which can modulate the functional activity of the pathogen expressing that antigen versus those which cannot. Even when analysing antibodies of different immunoreactivities, such methods cannot identify which of these antibodies may additionally modulate pathogen functioning. The analysis of modulation of pathogen functioning is of particular importance where the antibody based clearance mechanisms which are up-regulated in an infected individual upon the induction of a B cell response are not the only therapeutic or prophylactic mechanism to provide a defence to the pathogen in issue. In particular, in relation to some disease conditions, the generation of antibodies which can interfere with the functioning of a pathogen provides significant protection above and beyond that normally provided by antibody based clearance mechanisms, alone.

In order to enable the detection, monitoring and identification of functionally inhibitory immunointeractive molecules, in particular antibodies, the inventors have designed an assay which screens both for specific immunoreactivity and modulation of pathogen functioning. This is achieved by conducting a relative analysis of the level of functional activity of a pathogen, subsequently to its culture with the biological sample of interest, expressing the native form of the antigen of interest versus that of a pathogen expressing a form of the antigen which would not be recognised by the antibody in issue. Specifically, where there is observed a lower level of functional activity in native pathogen cultures versus that observed in the epitopically distinct pathogen cultures, there is indicated the presence of antibody which is both immunoreactive with the antigen which was rendered epitopically distinct in the control cultures and inhibitory of one or more of the functional activities of the pathogen of interest. Where the results do not demonstrate a difference in the level of functional activity expressed by the pathogen in the two forms of cultures which are established, the results indicate that there is not present in the biological sample any antibody which is directed to the antigen of interest and which modulates the functioning of the pathogen. These results do not indicate, however, that there is not present antibody in the culture which does recognise that epitope but which antibody does not modulate the functioning of the pathogen.

Reference to a biological sample “contacting” a pathogen of interest should be understood as a reference to any method of facilitating the interaction of any one or more components of the biological sample with the pathogen, or molecules shed or secreted therefrom, such that coupling, binding or other association may occur. In this regard, it should be understood that the method of the present invention may be performed in vitro or in vivo. With respect to the in vitro application of this method, the biological sample and the pathogen of interest are paced in contact in an artificial medium, such as a culture dish or flask. However, to the extent that the method of the present invention is applied in vivo, the biological sample and the pathogen of interest will be placed in contact within a biological organism such as an animal. In this regard, it should be understood that the pathogen and the biological sample may be separately or simultaneously introduced to the animal model such that they contact one another within the animal. Alternatively, the pathogen and the biological sample may be placed into initial contact prior to their introduction to the host animal, for example such that only one administration need be made to the animal. This form of administration should also be understood to fall within the scope of “contacting” as defined herein. Means of achieving such contact would be well know to those of skill in the art.

Reference to an “epitopically native” form of the antigen should be understood to mean that the epitope which is recognised by the antibody of interest is expressed by the pathogen either in its native/wild-type form or in a form which comprises amino acid or other structural or non-structural differences which do not impact on the ability of the antibody to recognise and bind the epitope. Reference to an “epitopically distinct” form of the subject antigen should be understood to mean that the epitope which is recognised by the antibody of interest has been altered such that it is no longer recognised and bound by the antibody of interest. The subject alteration can be achieved by any one or more of a number of techniques which would be known to the person of skill in the art including, but not limited to:
(i) deletion of the epitope from the antigen;
(ii) replacement of the epitope with a homologous form of the epitope which is not recognised by the antibody of interest;
(iii) any one or more amino acid deletions, additions or substitutions to the subject epitope;

In accordance with the exemplification provided herein, which is directed to the identification of functionally inhibitory antibodies directed to the Plasmodium falciparum MSP-1₁₉ antigen, the MSP-1₁₉ antigen of the Plasmodium falciparum merozoite is replaced with a homologous form of the antigen which is not recognised by the antibodies of interest. Specifically, the epitopically distinct form of Plasmodium falciparum is a genetically engineered form which expresses the MSP-1₁₉ region from P. chabaudi, being the form of Plasmodium which infects mice. In this regard, the strain of Plasmodium falciparum which expresses the native form of MSP-1₁₉ and which is exemplified herein is the D10 strain. The P. falciparum parasites expressing divergent MSP-1₁₉ domains are transfected D10 strain parasites which express the P. chabaudi domain (D10-PcMEGF). Whereas DIO-PcMEGF is a form of Plasmodium falciparum in which the entire EGF domains from MSP-1₁₉ are replaced with those from P. chabaudi, the D10-PcM3′ strain of Plasmodium falciparum is one in which the Plasmodium parasite expresses a chimeric form of MSP-1₁₉ in which approximately three quarters of the two EGF-like domains that comprise MSP-1₁₉ are replaced with the equivalent domains from the divergent rodent malaria P. chabaudi.

Accordingly, in a preferred embodiment there is provided a method of detecting the presence of a functionally inhibitory antibody in a biological sample, which antibody is directed to Plasmodium falciparum MSP-1₁₉, said method comprising:
(i) contacting a Plasmodium falciparum schizont of strain D10-PcM3′ with said sample for a time and under conditions sufficient to facilitate immunointeraction;
(ii) contacting a Plasmodium falciparum schizont of the strain D10 with said sample for a time and under conditions sufficient to facilitate immunointeraction;
(iii) assessing the level of functional activity of the Plasmodium falciparum strains of step (i) and step (ii);
   wherein a decrease in the functional activity of the Plasmodium falciparum strain of step (ii) relative to the Plasmodium falciparum strain of step (i) is indicative of the presence of a functionally inhibitory antibody in said sample.

In another preferred embodiment, there is provided a method of detecting the presence of a functionally inhibitory antibody in a biological sample, which antibody is directed to Plasmodium falciparum MSP-1₁₉, said method comprising:
(i) contacting a Plasmodium falciparum schizont of strain D10 with said sample for a time and under conditions sufficient to facilitate immunointeraction;
(ii) contacting a Plasmodium falciparum schizont of strain D10-PcMEGF with said sample for a time and under conditions sufficient to facilitate immunointeraction;
(iii) assessing the level of functional activity of the Plasmodium falciparum strains of step (i) and step (ii);
   wherein a decrease in the functional activity of the Plasmodium falciparum strain of step (ii) relative to the Plasmodium falciparum strain of step (i) is indicative of the presence of a functionally inhibitory antibody in said sample.

To the extent that the method of the present invention is performed in vivo, the person of skill in the art must additionally give consideration to the strain/species of pathogen which is to be introduced to the selected host animal. For example, the in vitro model exemplified herein utilises P. falciparum strains which express either the native form of P. falciparum MSP-1₁₉ or all or part of the homologous and epitopically divergent P. chabaudi MSP-1₁₉ domain. However, the use of these strains of P. falciparum could only be utilised to perform an in vivo screening assay where the host animal which is utilised is one which P. falciparum could colonise. That is, many pathogens demonstrate species specificity. Accordingly, whereas it may be feasible to directly utilise these specific P. falciparum strains in some host animals, such as primates, it would not be possible to use them with a rodent animal model. Accordingly, where it is desired to perform the in vivo screening method described herein, the person of skill in the art must select, for use, a strain of pathogen which is able to infect the host animal of interest.

For example, and in keeping with the embodiments exemplified herein, an in vivo murine screening assay which is directed to identifying the presence of functionally inhibitory antibodies directed to P. falciparum MSP-1₁₉ could be performed utilising the P. chabaudi or the P. berghei species. These species are both known to colonise mice. In this regard, the “epitopically native” pathogen could be achieved be engineering a P. chabaudi or P. berghei parasite such that it expresses the P. falciparum MSP-1 ₁₉ domain. The “epitopically distinct” pathogen could be provided, for example, in the form of the wild type P. chabaudi or P. berghei which express the murine homolog of the P. falciparum MSP-1₁₉ domain, which form is not recognised by antibodies directed to the P. falciparum form of MSP-1₁₉. In light of the teachings herein, it is within the skill of the person of skill in the art to select, for both in vitro and in vivo application, appropriate species/strains of pathogen for use. Accordingly, it should be understood that in relation to one or both of the pathogens expressing the native and epitopically distinct form of the antigen in issue, the pathogen species from which the antigen is derived need not necessarily correlate with the species of the pathogen which is expressing that antigen. That is, all or some of the pathogens which are utilised in accordance with this method may be genetically altered chimaeras. It should also be understood that the functionally inhibitory antibody which forms the subject of analysis may be one which was generated in the mice (for example as a result of the testing of the immunogenicity of a vaccine) or it may have been administered to the mice before, after or together with the pathogen strain (for example where one might be seeking to test in vivo the antibody load present in a human serum sample).

Accordingly, in yet another embodiment there is provided a method of detecting the presence of a functionally inhibitory antibody in a population of mice, which antibody is directed to Plasmodium falciparum MSP-1 and which method is performed in vivo in said mice, said method comprising:
(i) introducing to at least one of said mice a wild-type Plasmodium berghei for a time and under conditions sufficient to facilitate immunointeraction;
(ii) introducing to at least one of said mice, other than the mosue of step (i), a Plasmodium berghei strain, which strain expresses the Plasmodium falciparum MSP-1 block 17C-terminal domain, for a time and under conditions sufficient to facilitate immunointeraction;
(iii) assessing the level of functional activity of the Plasmodia of step (i) and step (ii)
   wherein a decrease in the functional activity of the Plasmodium berghei of step (ii) relative to the Plasmodium berghei of step (i) is indicative of the presence of said functionally inhibitory antibody in said mice.

Most preferably, there is provided a method of detecting the presence of a functionally inhibitory antibody in a population of mice, which antibody is directed to Plasmodium falciparum MSP-1₁₉ and which method is performed in vivo in said mice, said method comprising:
(i) introducing to at least one of said mice a wild-type Plasmodium berghei for a time and under conditions sufficient to facilitate immunointeraction;
(ii) introducing to at least one of said mice, other than the mouse of step (i), a Plasmodium berghei schizont of the strain Pb-PfM19 for a time and under conditions sufficient to facilitate immunointeraction;
(iii) assessing the level of functional activity of the Plasmodium berghei strains of step (i) and step (ii)
   wherein a decrease in the functional activity of the Plasmodium berghei strain of step (ii) relative to the Plasmodium berghei strain of step (i) is indicative of the presence of said functionally inhibitory antibody in said mouse.

In further aspects of this embodiment said MSP-1 antigen is alternatively the block 2 N-terminal domain, AMA-1, MSP-2, MSP-3, MSP-4, MSP-5, RAP-2, RAP-1, EBA-175 or CSP.

Reference to “biological sample” should be understood as a reference to any sample of biological material derived from an animal such as, but not limited to, mucus, biopsy specimens, fluid which has been instructed into the body of animal and subsequently removed such as, for example, the saline solution extracted from the lung following lung lavage, serum, plasma or in vitro derived biological sample such as ascites fluid or tissue culture supernatant. To the extent that the method of the invention is performed in an in vivo model, the biological sample may be a sample, as detailed above, which is introduced to the animal model. Alternatively, where the animal model itself has undergone the induction of the immune response which is to be analysed in that animal, by virtue of the in vivo analysis method disclosed herein, it should be understood that the animal itself falls within the scope of the phrase “biological sample”. The biological sample which is tested according to the method of the present invention may be tested directly or may require some form of treatment prior to testing. For example, a biopsy sample may require homogenisation prior to testing. Further, to the extent that the biological sample is not in a liquid form (for example it may be a solid, semi-solid or a dehydrated liquid sample), it may require the addition of a reagent, such as a buffer, to mobilise the sample prior to application of the method of the invention. This sample may also be treated in terms of undergoing a partial purification step, viral inactivation step or other form of pre-treatment.

The person of skill in the art would understand that steps (i) and (ii) can be performed in either order or simultaneously, the objective of these steps being to provide the framework within which a relative analysis of the functional readout of step (iii) is facilitated. In this regard, it should be understood that steps (i) and (ii) need not even be performed at substantially the same time. For example, in some instances, which will be obvious to the person of skill in the art, steps (i) and (ii) may be performed days, or even weeks, apart with the results subsequently analysed relative to one another. Further, it is feasible that results obtained in relation to step (i) or step (ii) could be utilised as a standard set of control results, thereby enabling the person of skill in the art to perform, in some suitable circumstances which would be obvious to the person of skill in the art, only one of step (i) or (ii), subsequently to which the results obtained thereon are analysed relative to the previously obtained “standard” result.

The method of the present invention is predicated on facilitating the immunointeraction of an antibody with an antigen. By “immunointeraction” is meant that interaction, binding or other form of association of the antibody of interest with the antigen of interest occurs. It would be well known to those skilled in the art as to how this could be achieved at either the in vitro or in vivo levels.

Determining the nature of the functional activity which should form the basis of assessment in relation to any given antigen or immunointeractive molecule would be determinable by the person of skill in the art based on the common general knowledge. It should be understood that assessment of the “level” of functional activity is intended to encompass assessment of the nature or occurrence of a particular functional activity. Means of assessing the level of functional activity of the pathogen would be well know to the person of skill in the art and could be achieved by any convenient means. Further, it should be understood that the method of the present invention can be adapted to screen for the subject antibodies at the qualitative and/or the quantitative levels.

In the method of the invention exemplified herein, the antibodies which are screened for are human antibodies directed to the P. falciparum MSP-1₁₉, which antibodies prevent invasiveness of the P. falciparum merozoite. In accordance with this objective, one embodiment of the invention is directed to screening human serum samples in an in vitro assay where ring-stage parasites (D10 parasites being the parasitic strain which expresses the native form of MSP-1₁₉ and D10-PcM3′ or D10-PcMEGF which express some or all of the P. chabaudi MSP-1₁₉ antigens) are synchronised and allowed to mature through to the trophozoite/schizont stage. These mature parasites are then co-cultured with red blood cells and the serum sample of interest. These cultures are incubated for a time and under conditions sufficient to allow for schizont rupture, merozoite invasion and antibody binding. The assessment of functional activity of the Plasmodium falciparum strains tested herein is assessed by three mechanisms as follows:
(i) Microscopy analysis: Smears are made of the cultured parasites and the number of ring-stage parasites per red blood cells is determined. The mean parasitemia can then be calculated and expressed as a percentage of the mean parasitemia observed in parallel cultures.
(ii) [³H]hypoxanthine uptake assay: Following culture of the parasites, the culture medium is removed and replaced with hypoxanthine-free medium supplemented with [³H]hypoxanthine. Following a further 24 hours of culture the mature parasites are frozen and thawed in order to effect lysis of the infected red blood cells. Samples are then transferred to glass fibre filters via a cell harvester and quantitated using a scintillation counter.
(iii) Co-cultivation assays: In these assays the epitopically native P. falciparum strain and the epitopically distinct P. falciparum strain are co-cultured subsequently to having been synchronised at ring stage and allowed to mature through to the trophozoite/schizont stage. They are cultured together at an equal ratio in the presence of serum. Parasites are smeared at the trophozoite/schizont stage and assessed by indirect immunofluorescence.
(iv) FACS detection: For example cultures are set up as for microscopy analysis. The parasites are allowed to mature a further 24 hours (ie. 48 hours after set-up) and are then labelled with hydroethidine (HE). HE is incorporated into the DNA of viable parasites only and can be detected by flow cytometry. HE is added to parasites, incubated at 37° C. in the dark for 20 minutes, diluted with buffer and pelleted. Pellets are resuspended for FACS.

The development of a method of detecting functionally inhibitory immunointeractive molecules now facilitates its application to a range of situation including, but not limited to:
(i) predicting the immune status of individuals who have been previously infected with a pathogen;
(ii) predicting the immune status of individuals vaccinated with vaccines designed to administer a specific antigen. This is particularly valuable in relation to clinical trials;
(iii) determining the relative contribution of an antibody of specific immunoreactivity to the total inhibitory antibody elicited by combination vaccines which include two or more antigens, one of which is the antigen to which the subject antibody is directed.
(iv) the assessment of human vaccines in a mouse model system. This is particularly important with respect to assessing the efficacy of different forms of an antigen which are proposed to be utilised in a vaccine (eg. antigens which have been prepared utilising different methodologies) and assessing vaccine potency (which for some vaccines reduces over time when stored);
(v) assessment of the protective potential of an antibody of interest which is present in human serum (for example, of infected or vaccinated individuals). This requires passive transfer of human antibodies into mice which are subsequently infected with the epitopically distinct pathogen in issue, as detailed hereinbefore.
(vi) determination of the importance of functionally inhibitory antibodies to clinical protection. This is of particular importance since it will reveal much about the mechanism of protection which occurs in immune individuals.

Accordingly, in another aspect the present invention is directed to a method of assessing the nature of an immune response to an antigen in accordance with the methods defined hereinbefore. The term “nature” should be understood in its broadest sense as a reference to any one or more qualitative and/or quantitative aspects of an immune response. As detailed above, this provides a means of assessing an immune response to a pathogen in accordance with points (i)-(vi), above.

In yet another aspect, the present invention extends to the pathogens defined herein.

Accordingly, yet another aspect of the present invention is directed to an isolated pathogen, which pathogen expresses a non-wild-type form of one or more antigens derived from said pathogen.

More particularly, the present invention provides an isolated malaria pathogen, which pathogen expresses a non-wild-type form of one or more antigens derived from said pathogen.

It should be understood that reference to the terms “pathogen” and “malaria” have the same meaning as hereinbefore defined. Similarly, the phrase “antigen(s) derived from said pathogen” should be understood to have the same meaning as the previously defined phrase “pathogen derived antigen”. Also, as detailed hereinbefore, the antigen may be one which is permanently or transiently expressed in either a constitutive or inducible manner. This may largely depend on the life-cycle stage of the pathogen at any given point in time.

Reference to a “non-wild-type” form of an antigen should be understood as a reference to the form of the subject antigen which differs from the form expressed by the wild-type form of the pathogen. In this regard, the non-wild-type form will generally differ from the wild-type form by virtue of the amino acid sequence of the subject antigen. However, the present invention is not limited in this regard and any other form of change which would render an antigen “non-wild-type” is encompassed in this definition. In the context of the present invention, the non-wild-type form of the antigen will generally correspond to a homologous form of the antigen. For example, to the extent that the method of the present invention is applied to detecting the generation of antibodies in a mouse, directed to a human pathogen, one would utilise a form of the pathogen which can colonise and replicate in mice (since the assay is to be performed in mice) but wherein the viability in those mice of the wild-type form of the pathogen is analysed relative to a murine form of the pathogen which has been engineered to express the human version of the antigen to which the antibodies have been raised. In this regard, and as detailed hereinbefore, the murine form of the parasite which expresses the human homolog of the antigen to which antibodies may have been raised corresponds to the form of pathogen expressing an “epitopically native form” of the antigen since this is the form of antigen against which it was desired to raise antibodies. The wild-type form of the pathogen should be understood to express the “epitopically distinct form” of the antigen since it expresses a form of antigen against which the antibodies were not directed. Accordingly, reference to an antigen being “epitopically distinct” versus “epitopically native” is assessed relative to the form of antigen against which the presence of the functionally inhibitory antibody is being assessed. It should also be understood, however, that to the extent that the use of “wild-type” forms of pathogens are defined in the methods hereinbefore, a pathogen will satisfy that this definition provided it is “immunologically” wild-type. That is, that the antigen region in issue is not immunogenic in the species to which it is administered. Accordingly, some small changes to the subject “antigen” region may not change its immunogenicity and therefore render those pathogens effectively useful as “wild-type” pathogens.

Preferably, the present invention provides an isolated Plasmodium, which Plasmodium expresses a non-wild-type form of MSP-1.

More preferably, said MSP-1 is the block 17 C-terminal domain or the block 2 N-terminal domain of MSP-1.

Most preferably, said Plasmodium is Plasmodium berghei expressing the Plasmodium falciparum form of the MSP-1₁₉ antigen.

Even more preferably, said Plasmodium berghei is the Pb-PfM19 strain.

In another embodiment, the present invention provides an isolated Plasmodium pathogen expressing a non-wild-type form of one or more antigens derived from said pathogen, which antigens are selected from the list of:
(i) the apical membrane domain (AMA-1)
(ii) merozoite surface protein 2, 3, 4 and/or 5 (MSP-2, MSP-3, MSP-4 and/or MSP-5)
(iii) rhoptry associated protein 2 (RAP-2)
(iv) erythrocyte binding antigens (EBA-175)
(v) circumsprozoite antigen (CSP)

In accordance with this preferred embodiment, still more preferably the subject malaria pathogen is a Plasmodium pathogen and still more preferably a Plasmodium falciparum pathogen, Plasmodium berghei pathogen and/or Plasmodium chabaudi pathogen.

In yet another aspect, the present invention extends to the pathogens defined herein when used in accordance with the method of the present invention.

Further features of the present invention are more fully described in the following non-limiting Examples.

### EXAMPLE 1

### Antibodies Against Merezoite Surface Protein (MSP)-1₁₉ are a Major Component of the Invasion Inhibitory Response in Individuals Immune to Malaria

Materials And Methods

Plasmids

Construction of the plasmids pFfM3′ and pPcM3′ has been described previously (O'Donnell, R. A. et al. 2000 supra). The plasmid pPcMEGF was constructed by the insertion of a 1,200-bp XhoI fragment into the unique XhoI site of a plasmid pHC2 (Triglia, T., Healer, J., Caruana, S. R., Hodder, A. N., Anders, R. F., Crabb, B. S. and Cowman, A. F. (2000) Mol. Microbiol. 38:706-718). This target fragment comprises a 900-bp internal region of the P. falciparum MSP-1 gene fused in frame to the MSP-1₁₉ region of P. chabaudi. The fragment was generated by PCR amplification from P. falciparum (D10) and P. chabaudi (adami DS) genomic DNA (gDNA) using the oligonucleotide pairs Pf#1 5′-ATTTCTCGAGAATCCGAAGATAATGACG-3′ (<400>1), PfEGF-R 5′-GAAACATCCAGCATTTTCTGGAAGTTTGTTCCTATGCATTGGTGTTGTGAAATG-3′ (<400>2). The resulting amplicons were sewn together via PCR for insertion into pHC2. The XhoI sites are shown in bold.

Parasite Culture and Transfection Procedures

P. falciparum line D10 was cultivated and synchronised as per standard procedures (Larnbros, C. and Vanderberg, J. P. (1979) J. Parasitol. 65:418-420; Trager, W. et al. 1976). Ring-stage parasites (˜5% parasitemia) were transfected with 50-100 μg of CsCl-purified plasmid DNA as described previously (Crabb, B. S. and Cowman, A. F. (1996) Proc. Natl. Acad. Sci. USA. 93:7289-7294; Crabb, B. S., Triglia, T., Waterkeyn, J. F. and cowman, A. F. (1997) Mol. Biochem. Parasitol. 90:131-144) but using the electroporation conditions as described by Fidock and Wellems (Fidock, D. A. and Wellems, T. E. (1997) Proc. Natl. Acad. Sci. USA. 94: 10931-10936). After transfection and initial selection using 0.1 μM pyrimethanine for ˜4 weeks, parasites were subjected to repeated cycles of 1 μM pyrimethamine for 3 weeks proceeded by removal of the drug for 3-4 weeks. gDNA was extracted from mixed trophozoit/schizont stage parasites as described previously (Coppel, R. L. and Biano, A. E., Culvenor, J. G., Crewther, P. E., Brown, G. V., Anders, R. F. and Kemp, D. J. (1987) Mol. Biochem. Parasitol. 25:73-81), and Southern blot analysis was carried out using standard procedures (Sambrook, J., Fritsch, E. F. and Maniatis, T. (1989) Molecular Cloning: A Laboratory Manual. Cold Spring Harbor Laboratory Press, New York).

Western Blot Analysis

Parasite proteins were obtained from extracted enriched schizont or merozoite preparations and separated using 7.5 and 12% SDS-PAGE nonreducing gels, respectively, and transferred to PVDF membranes (Amersham Pharmacia Biotech). membranes were probed with either mouse ascitic fluid containing 4H9/19, a monoclonal antibody specific for P. falciparum MSP-1₁₉ (Cooper, J. A. et al. 1992 supra), diluted 1:80,000 or rabbit *PcM19 polyclonal antibodies diluted 1:2,500 that are specific for P. chabaudi MSP-1₁₉ (O'Donnell, R. A. et al. 2000 supra). Horseradish peroxidase-conjugated rabbit anti-mouse (Dako) or sheep anti-rabbit (Silenus) Igs were used for detection, and bands were visualised by enhanced chemiluminescence (NEN Life Science Products).

Indirect Immunofluorescence

For indirect immunofluorescence assay (FA), D10-PfM3′ and D10-PcMEGF schizont-stage parasites were incubated with a mixture of 4H9/19 ascitic fluid and *PcM19 sera diluted 1:4,000 and 1:1,000, respectively. After incubation in the presence of a mixture of FITC-conjugated sheep anti-mouse and rhodamine-conjugated goat anti-rabbit Igs (Dako), both diluted 1:150, parasites were visualised by fluorescence microscopy. The same fields were photographed using filters to detect the FITC or rhodamine fluorochromes.

Sera

The Papua New Guinean sera used were collected in the Madang Province from adults living in and around Madang town in 1980-82 (denoted PNG-M sera) and from adults currently living on Bagabag Island (denoted PNG-B sera). both locations have high prevalence rates of P. falciparum (over all rates of 25.7 and 24%, respectively, with the highest rates observed in 1-9 year-old children in both communities) that are indicative of intense transmission (Cattani, J. A., Tulloch, J. L., Vrbova, H., Jolley, D., Gibson, F. D., Moir, J. S., Heywood, P. F., Alpers, M. P., Stevenson, A. and Clancy, R. (1986) Am. J. Trop. Med. Hyg. 35:3-15). Transmission in these localities is perennial with similar rates in the wet and dry seasons.

To generate P. chabaudi immune mouse sera (Pc immune), six 7-week-old C57BL/6 male mice were injected intraperitoneally with 5×10³ P. chabaudi (adami DS)-infected RBCs and rechallenged at 3 weeks with the same dose. At weeks 7 and 21, mice were administered a higher challenge of 10⁴ P. chabaudi-infected RBCs before serum collection at week 24.

MSP-1₁₉ Glutathione S Transferase Fusion Proteins

The DNA sequence corresponding to the MSP-1₁₉ fragment lacking the glycosylphosphatidylinositol anchor sequence (amino acids Asn 1631-Ser 1723, according to reference 27) was amplified from P. falciparum D10 or HB3 gDNA (which contains the MAD20 or K1 MSP-1₁₉ alleles, respectively; reference 27) using the oligonucleotides: PfM19f 5′-CGCGGATCCAACATTTCACAACACCAATGCG-3′ (<400>3) and PfM19r 5′-GGAAGATCTTAACTGCAGAAAATACCATCGAAAAG-3′ (<400>4). The resulting PCR products were ligated into the BamHI site of pGEX-4T-1, expressed as glutathione S transferase (GST) fusion proteins in Escherichia coli (Smith, D. B. and Johnson, K. S. (1988) Gene 67:31-40) and purified using glutathine-sepharose as described by the manufacturer (Amersham Pharmacia Biotech). GST alone was produced using the pGEX-4T-1 plasmid.

ELISA

Antibodies reacting with recombinant P. falciparum MSP-1₁₉ and P. chabaudi MSO-1₁₉ were detected by ELISA. Microtitre plates (Dynex) were coated overnight at 4° C. with 0.5 μg/ml recombinant protein diluted in carbonate buffer (0.015 M Na₂CO₃, 0.035 M NaHCO₃, pH 9.6). Plates were washed three times with PBS containing 0.05% Tween 20 (PBST), blocked for 2 hours at 37° C. with PBST containing 10 mg/ml BSA, and washed again. Sera diluted in PBST containing 10 mg/ml BSA, and washed again. Sera diluted in PBST containing 5 mg/ml BSA was then added to the plates (50 μl), which were then incubated further for 1 hour at 37° C. After washing with PBST, horseradish peroxidase-conjugated sheep anti-human IgG (1:4,000; Silenus), rabbit anti-mouse IgG (1:5,000), or sheep anti-rabbit IgG (1:2,500; Dako) were added to the plates, incubated for 1 hour at 37° C. and, after washing with PBST, developed with H₂O₂ and 3,3′, 5,5′-tetramethylbenzidine dihydrochloride for 10 min at room temperature. The reaction was stopped by the addition of 20 μl 2.5 M H₂SO₄ and plates were read at 450 nm. All human and mouse sera were tested in duplicate at three dilutions (1:1,000, 1:3,200, and 1:10,000) against GST-PfM19, GST-PcM19, and GST alone. The mean optical density (OD) value derived from GST alone was subtracted from the mean OD obtained for each GST fusion protein. Values at a 1:3,200 dilution were considered most likely to be on the slope of a titration curve, hence, these values are represented here.

Inhibition of Invasion Assays

Ring-stage parasites were synchronised by sorbitol lysis twice at 4 hour intervals and then allowed to mature through to trophozoite/schizont stages. the purified parasites were adjusted to 4% hematocrit with 0.5-2% infected RBCs and aliquots of 50 μl placed into the wells of a 96-well tray. An equal volume of serum, prediluted 1:10 in culture media, was added (resultant hematocrit of 2%) and cultures incubated for ˜26 hours to allow for schizont rupture and merozoite invasion. Note that when two parasite lines were being compared the same batch of prediluted serum was added to each line. For the microscopy analysis, smears were made of the duplicate wells, stained with Giemsa, and the number of ring-stage parasites per 500 RBCs were determined for each well. The mean parasitema from duplicate wells was calculated and this was expressed as a percentage of the mean parasitemia observed in parallel cultures of each parasite line in the presence of pooled human nonimmune sera (HNIS). For the [³H]hypoxanthine uptake assay, media was removed from triplicate wells at ˜24 hours after cultivation and replaced with hypoxanthine-free media supplemented with [³H]hypoxanthine (10 μCi/ml). A further 24 hours later, cultures of mature parasites were frozen and thawed to lyse infected RBCs. Samples were transferred to glass fiber filters via a cell harvester and quantitated using a scintillation counter. Statistical analysis for address whether the mean invasion rates of these sera were the same between two lines was performed using a two-sample Student's t test assuming unequal variances.

Co-cultivation Assays

For co-cultivation, D10-PfM3′ and D10-PcMEGF ring-stage parasites were doubly synchronised as described above and then cultured together at an equal ratio in the presence of pooled sera. Sera was pooled on the basis of either appearing to contain significant proportions of anti-MSP-1₁₉ inhibitory antibodies (pool 2) or having a less inhibitory effect between the two parasite lines (pool 1). The pools included the following sera: (PGN-B pool 1) 8, 247, 332, and 962; (PNG-B pool 2) 413, 604, 614, and 954; (Pc-immune pool 1) 2 and 4; and (Pc-immune pool 2) 1, 3, 5, and 6. Additional controls included a HNIS pool and a *PcM19 rabbit serum. Parasites were smeared at the trophozoite/schizont stage (ie., every 2 days beginning at day 1) and assessed by indirect IFA using a mixture of 4H9/19 and *PcM19 as described above. For each of the triplicate slides, FITC (DIO-PfM3′) and rhodamine (D19-PcMEGF) were observed and counted in 16 individual fields that each contained at least 10 mature (pigmented) parasites. In total, between 400 and 1,400 mature stage parasites were counted for each co-cultivation sample.

Results

Replacement of Complete EGF Domains of P. falciparum MSP-1₁₉ with those from P. chabaudi

The aim of this study was to generate a P. falciparum line that possesses an antigenically distinct MSP-1₁₉ domain and to investigate whether this line differs from parental parasites in its susceptibility to inhibition by sera from malaria-immune individuals. We have described previously the construction of a parasite line D10-PcM3′ which expresses an MSP-1 chimera in place of the endogenous molecule (O'Donnell, R. A. et al. 2000 supra).

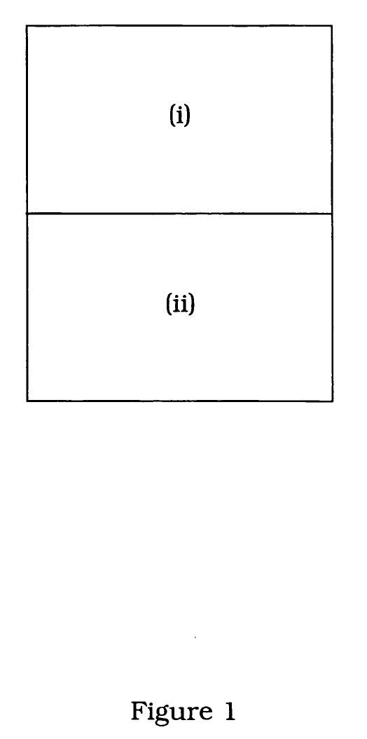

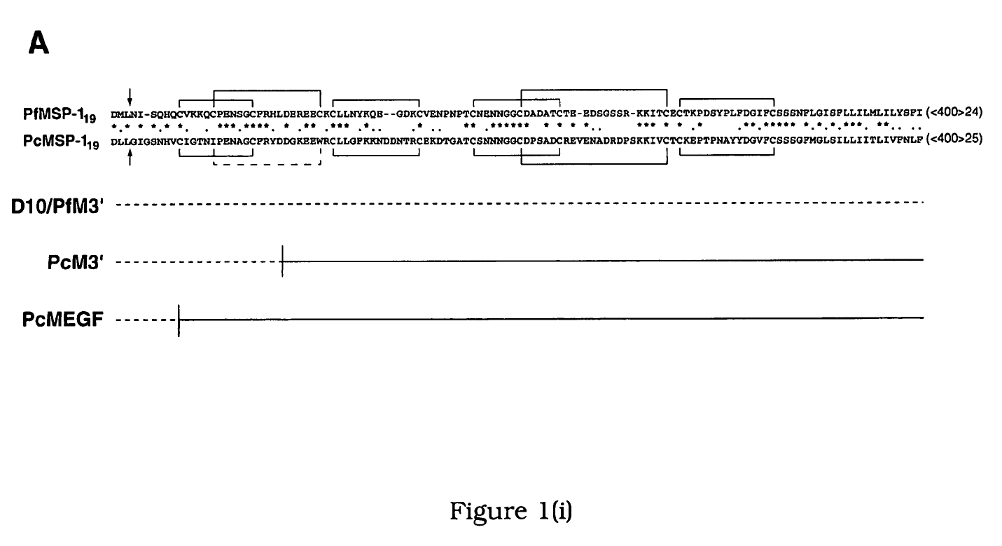

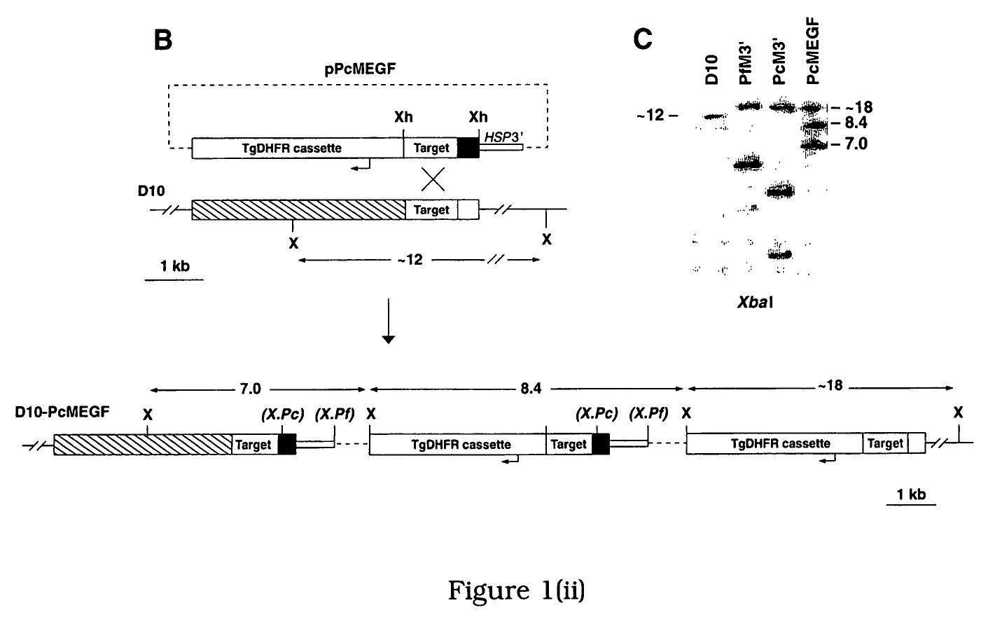

This chimera incorporates ˜¾ of the two EGF-like domains that comprise MSP-1₁₉ from the divergent rodent malaria P. chabaudi (FIG. 1A). We also constructed a control transfectant D10-PfM3′ which expresses endogenous MSP-1 (FIG. 1A; reference 11). These transfected lines displayed no observable phenotypic differences to parental D10 parasites revealing that the function of most of MSP-1₁₉ is conserved across divergent Plasmodium species. Here we describe transfection of a plasmid, pPcMEGF, designed to replace the entire EGF domains from MSP-1₁₉ with those from P. chabaudi (FIG. 1). Upon transfection and drug cycling, pPcMEGF was shown to have integrated into the MSP-1 gene. The transfected population, D10-PMEGF, was cloned and two randomly selected clones (D10-PcMGF.1 and D10-PcMEGF.2) were analyzed further. Southern blot analysis showed the plasmid had integrated into the target site through the expected recombination event replacing the entire endogenous P. falciparum MSP-1₁₉ EGF domains with those from P. chabaudi. This line would be distinguished from both D10-PfM3′ and D10-PcM3′ by restriction endonuclease digestion with XbaI (FIG. 1, B and C).

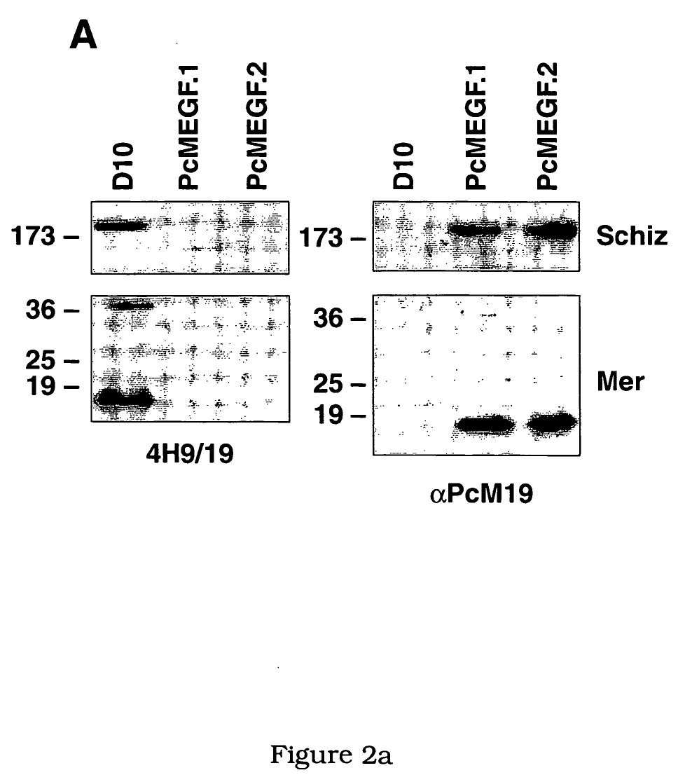

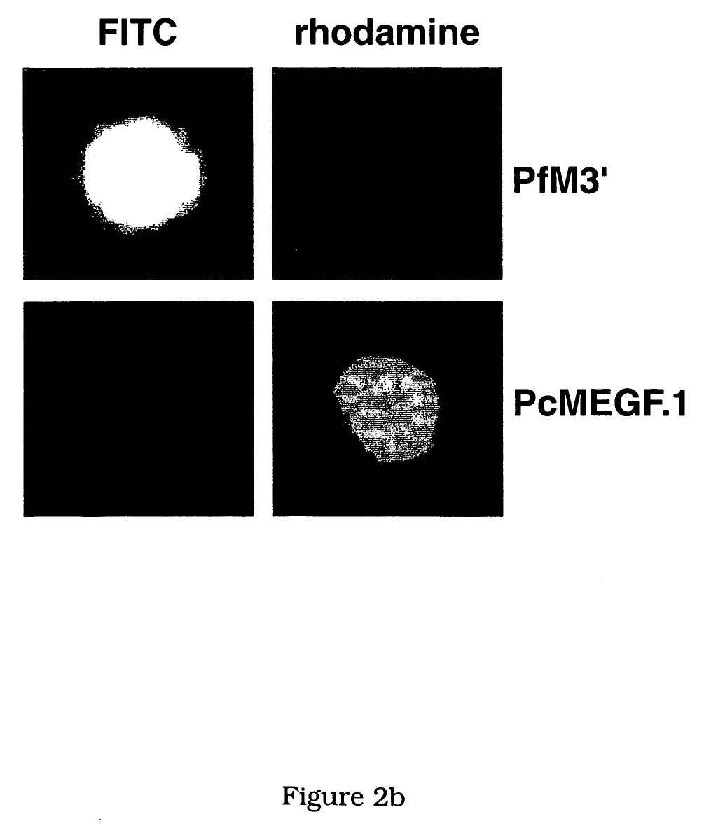

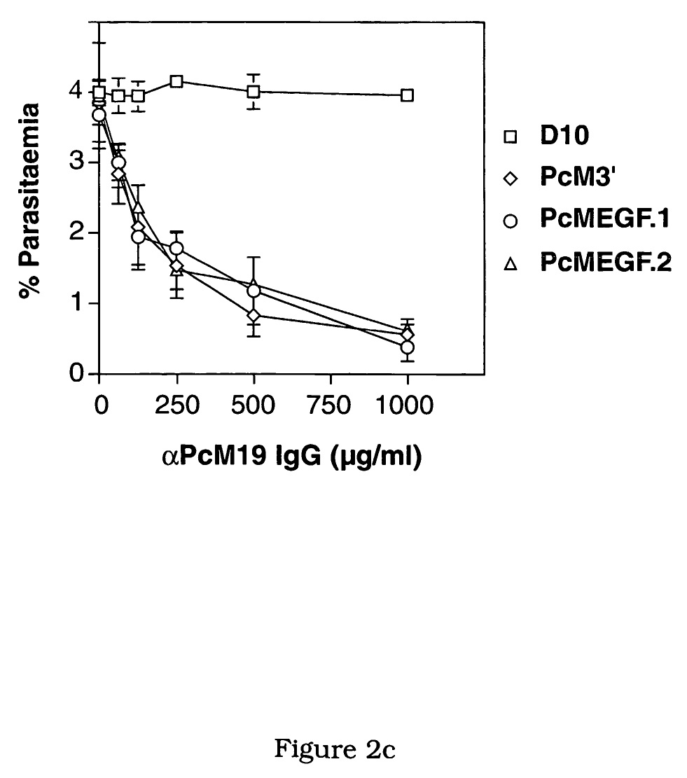

To determine if the chimeric MSP-1 was expressed in D10-PcMEGF parasites, extracts of mature schizonts and free merozoites were examined by immunoblot analysis (FIG. 2A). Bands corresponding to endogenous full-length MSP-1 (˜200 kD) and MSP-1₁₉ (˜18 kD) were detected in parental D10 schizonts and merozoites, respectively, using the P. falciparum-specific antibody 4H9/19 (Cooper, J. A. et al. 1992 supra). No reactive bands were observed in extracts from the two D10-PcMGF clones. Conversely, when replicate immunoblots were probed with a rabbit antiserum specific for P. chabaudi MSP-1₁₉ (*PcM 9; reference 11), species corresponding to both forms of MSP-1 were observed in the D10-PcMEGF extracts but not in parental D 10 (FIG. 2A). The larger band (40 kD) in the merozoite samples is consistent with the presence of the primary MSP-1 processing product, MSP-1₄₂. The localisation of the MSP-1 chimera was assessed by an IFA (FIG. 2B). D10-PfM3′ and D10-PcMEGF parasites were incubated with a mixture of mouse 4H9/19 and rabbit *PcM19 antibodies followed by FITC-labelled anti-mouse (to detect endogenous MSP-1) and rhodamine-labelled anti-rabbit (to detect the MSP-1 chimera) IgG. “Grape-like” fluorescence was observed in both lines indicative of merozoite surface labelling. D10-PcMEGF parasites showed only rhodamine fluorescence supporting the absence of endogenous MSP-1₁₉ expression in this line. Fluorescence was also observed in ring-stage parasites indicating that the P. chabaudi MSP-1₁₉ domain is carried into the newly invaded RBCs in D10-PcMEGF parasites as has been described for P. falciparum MSP-1₁₉ (data not show; references 11 and 12).

To ensure that the chimeric MSP-1 was functional, an in vitro inhibition of invasion assay was carried out (FIG. 2C). Mature stage parasites from parental D10, D10-PcM3′, and two clones from D10-PcMEGF were incubated in the presence of *PcM19 IgG. These antibodies specifically inhibited RBC invasion of D10-PcMEGF and D10-PcM3′ parasites in a dose-dependent manner but had no effect on parental D10. These results are consistent with the correct expression, processing, localisation and functioning of the expected hybrid MSP-1 molecule in D10-PcMEGF parasites. This also reveals that the complete EGF domains of MSP-1₁₉ are functionally conserved across distantly related Plasmodium species.

Invasion-inhibition of Transfected P. falciparum Parasites by Immune Sera Reveals an Important Role for MSP-1₁₉ specific Antibodies

The availability of parasite lines that are identical except for the presence of antigenially distinct MSP-1₁₉ domains provided a unique opportunity to address the relative importance of MSP-1₁₉ antibodies to invasion-inhibition by immune sera. Sets of human sera were obtained from adults in two malaria endemic areas in Papua New Guinea that have intense transmission rates of P. falciparum (PNG-M and PNG-B). The majority, if not all, of these individuals are likely to be clinically immune to P. falciparum malaria. Sera from six C57BL/6 mice that had been repeatedly infected with P. chabaudi were also generated (Pc-immune sera). To determine the presence and the specificity of MSP-1₁₉ antibodies, each human and mouse serum was tested in ELISA against recombinant forms of P. falciparum MSP-1₁₉ (GST-PfM19) and P. chabaudi MSP-1₁₉ (GST-PcM19). All PNG-B sera (47/47) and most PNG-M sera (27/33) reacted against GST-PfM19 while only five human sera (all from PNG-B) showed detectable cross-reactivity with GST-PcM19 (Table 1). The OD₄₅₀ values against GST-PcM19 of these five cross-reactive PNG-B sera ranged from 0.277 to 0.900 (mean=0.451). The remaining 42 PNG-B serum samples had OD₄₅₀ values against GST-PcM19 below 0.113.

The six mouse sera showed no reactivity to GST-PFM19 but each showed strong reactivity with GST-PcM19. These results reveal that MSP-1₁₉ antibodies were generated in response to infection with either P. falciparum or P. chabaudi and that these were mostly highly specific for the homologous MSP-1₁₉ domain. The P. falciparum MSP-1₁₉ sequence of GST-PFM19 represented the “MAD20” allele; however, each serum was also tested in parallel with a GST-MSP-1₁₉ fusion protein comprising “KI/Wellcome” allelic sequence (Miller, L. H., Roberts, T., Shahabuddin, M. and McCutchan, T. F. (1993) Mol. Biochem. Parasitol. 59:1-14; Tanabe, K., Mackay, M., Goman, M. and Scaife, J. G. (1987) J. Mol. Biol. 195:273-287). The OD readings against this fusion protein were very similar to those obtained for the “MAD20” allele across all PNG-B and PNG-M sera (R²=0.914). This cross-reactivity between alleles is consistent with the finding of others (Egan, A. F., Chappel, J. A., Burghaus, P. A., Morris, J. S., McBride, J. S., Holder, A. A., Kaslow, D. C. and Riley, E. M. (1995) Infect. Immun. 63:456-466).

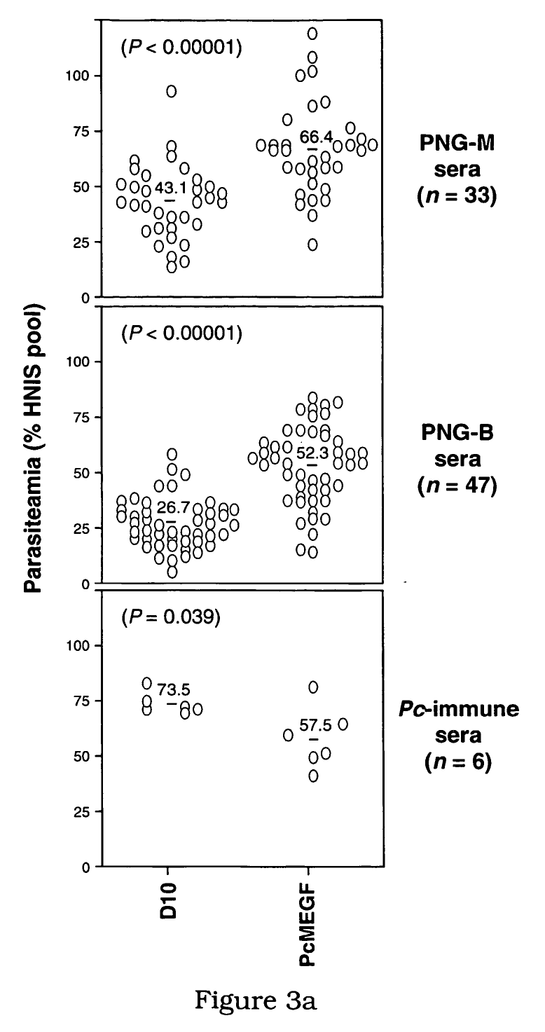

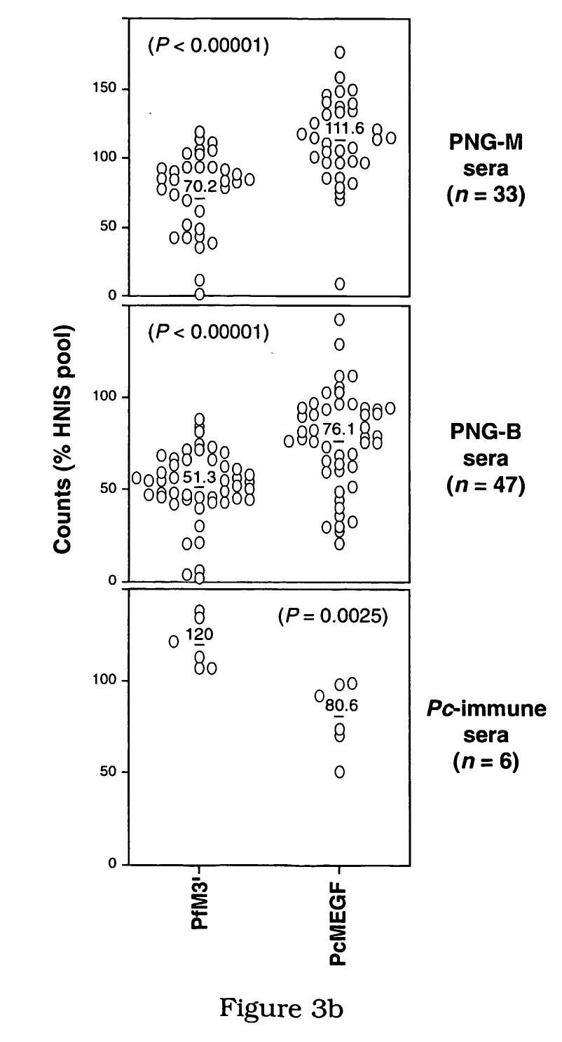

Preliminary experiments in our laboratory had indicated that D10-PcMEGF were relatively resistant to inhibition by human sera from malaria-immune individuals. To explore this more thoroughly, all PNG-M, PNG-B, and Pc-immune mouse sera were assessed for their ability to inhibit invasion of D10 and D10-PcMEGF merozoites in a microscopy-based invasion inhibition assay. All sera were tested in the one assay with the same parasite preparations (assay 1; FIG. 3A). PNG-B sera were relatively effective at inhibiting invasion of parental D10 parasites with a mean invasion of 26.7%. PNG-M sera were generally less inhibitory of D10 parasites (43.1%). The difference between PNG-B and PNG-M sera, both in invasion-inhibition and total MSP-1₁₉ antibodies (Table I), may simply reflect a loss of potency of PNG-M sera over relatively long-term cryopreservation period (˜20 years) although this was not explored further.

Strikingly, we found that both PNG-B and PNG-M sera were generally much less effective at inhibiting the invasion of 10-PcMEGF merozoites (FIG. 3A). Here, mean invasion rates of 52.3 and 66.4% were obtained for PNG-B and PNG-M, respectively, which in both cases was ˜25% higher than that obtained for D10. In contrast, the Pc-immune sera was more effective at inhibiting D10-PcMEGF (mean invasion rate 57.5%) than parental D10 (mean invasion 73.5%). In each case, the difference in the mean invasion rate was either significant or highly significant (FIG. 3A).

In an attempt to independently confirm these result, the inhibitory potential of these sera was tested by a different assay that utilizes [³H]hypoxanthine uptake as a measure of parasite growth (assay 2; FIG. 3B). In this assay, D10-PfM3′ was used as the parental control instead of D10 and again all sera were tested together in the one assay. The results were similar ot those obtained in assay 1 with D10-PfM3′ parasites more susceptible than D10-PcMEGF to inhibition by PNGOM and PNGOB sera. Again, as in FIG. 3A, D10-PcMEGF parasites were more susceptible than D10-PfM3′ to inhibition by Pc-immune sera. In each case, the difference in the mean invasion rates was highly significant. These results are consistent with a major role for MSP-1₁₉ antibodies in invasion/growth inhibition by malaria immune sera.

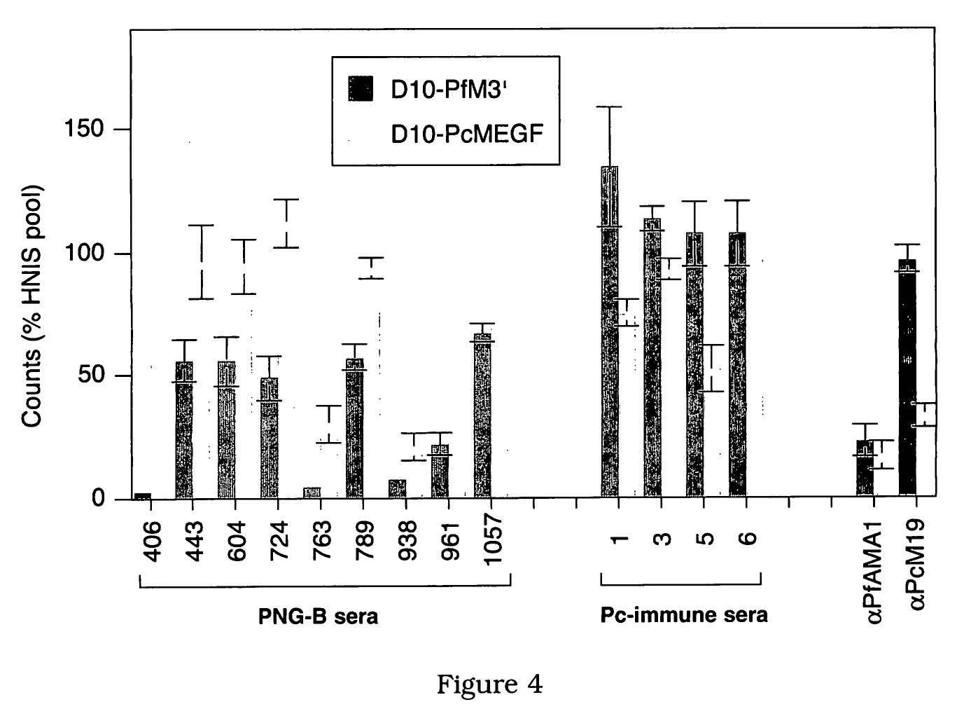

FIG. 4 shows inhibition results (from assay 2) that are representative of the data obtained for individual sera. Although some individual human sera did not appear to contain high levels of P. falciparum MSP-1₁₉-specific inhibitory antibodies (eg. 938, 961, and 1,057), a major proportion of the invasion-inhibitory component of other samples was directed against MSP-1₁₉ (eg. 406, 604, 724). Most human samples (59/80) showed some level of P. falciparum MSP-1₁₉-specfic inhibitory antibodies in either assay 1 or 2. All Pc-immune sera had detectable levels of P. chabaudi MSP-1₉-specific inhibitor antibodies in either assay 1 or 2. Results for the two control sera used in assay 2 are also shown (FIG. 4). the first was a polyclonal rabbit anti-P. falciparum AMA-1 IgG (Hodder, A. N., Crewther, P. E. and Anders, R. F. (2001) Infect. Immun. 69:3286-3294) used at a concentration of 250 μg/ml and the second was *PcM19 purified IgG used at a concentration of 750 μg/ml. both lines were equally susceptible to inhibition by *AMA-1 IgG, whereas only D10-PcMEGF was inhibited with *PcM19.

We also examined if there was any relationship between the amounts of MSP-1₁₉-specific invasion-inhibitory antibody and total MSP-1₁₉ IgG. MSP-1₁₉ invasion-inhibitory antibody in each human serum was calculated from microscopy-based assay by subtracting the percentage of invasion for D10-PfM3′ from the value obtained with D10-Pc-MEGF. These values showed no correlation with the OD₄₅₀ readings obtained for each serum against GST-PfM19 antigen (R²=0.013 and 0.0003 for PNG-M and PNG-B sera, respectively). However, it should be noted that four of the six PNG sera that were negative for GST-PfM19 antibodies (at a 1:3,200 dilution) in ELISA (Table I) also showed no detectable levels of MSP-1₁₉-specific invasion-inhibitory antibodies. The amount of P. chabaudi MSP-1₁₉-specific inhibitory antibody present in individual Pc-immune sera also showed no relationship to total P. chabaudi MSP-1₁₉-specific IgG (R²=0.0048).

Co-cultivation Assays in the Presence of Sera from Immune Individuals Support a Major role for MSP-1₁₉ Antibodies in Growth Inhibition

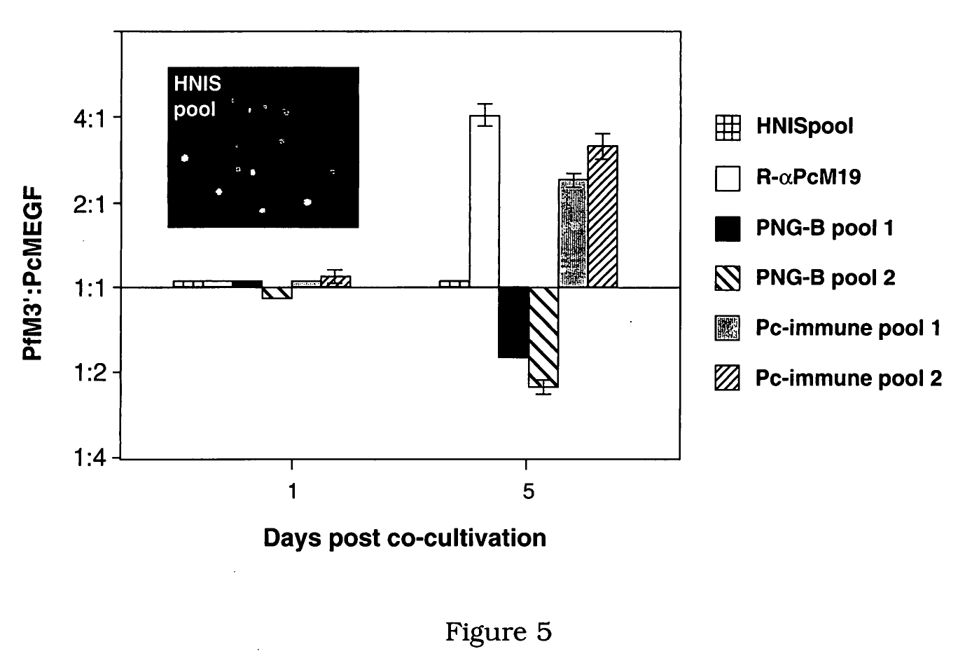

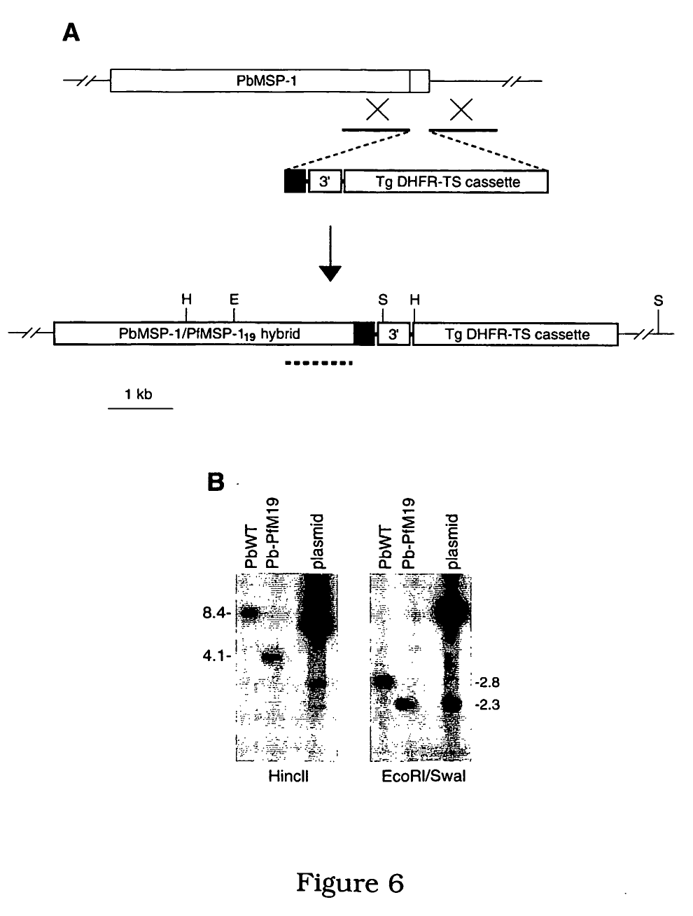

As an alternative means of addressing the specificity of the inhibitory antibodies in immune sera for MSP-1₁₉, D10-PfM3′ and D10-PcMEGF parasites were co-cultivated at an equal ratio in the presence of pooled sera. Several individual sera were pooled on the basis of the amount of anti-MSP-1₁₉-inhibitory antibody determined by the inhibition assays described above. Those with lower levels of MSP-1₁₉-specific inhibitory antibody comprised pool 1 while those with more apparent MSP-1₁₉-inhibitory antibody comprised pool 2. Parasites were detected by indirect IFA using a mixture of 4H9/19 and *PcM19 to detect D10-PfM3′ and D10-PcMEGF, respectively. The insert for FIG. 5 shows a typical field after incubation with pooled HNIS showing similar numbers of D10-PfM3′ (green) and D10-PcMEGF (red) parasites and illustrates the ease with which the two different lines were visualised in the mixed culture.

Red and green fluorescent parasites were counted after 1 and 5 days of co-cultivation in the presence of the different pooled sera. After 1 day of culture, where parasites were expected to have matured but not reinvaded fresh RBCs, no change in parasite ratio was observed with any sera. Co-cultivation in the presence of HNIS for 5 days also had no effect on the ratio of the two parasite lines confirming hat D10-PfM3′ and D10-PcMEGF have very similar growth rates (FIG. 5). However, incubation of the parasite mix with *PcM19 or Pc-immune sera had a dramatic effect on parasite ratio with the number of D10-PfM3′ parasites in 3-4-fold excess of D10-PcMEGF. In contrast, incubation with PNG-B-pooled sera had the opposite effect. It is important to note that the human and mouse sera pooled on the basis of possessing the most MSP-1₁₉-inhibitory antibody in the aforementioned assay (FIG. 3) also exhibited the greatest growth inhibition here.

### EXAMPLE 2

### Generation of Recombinant Parasites

Methods

Plasmids

The pHCl plasmid vector has been described (Crabb, B. S. et al. 1997 supra). XhoI insers for cloning into this plasmid were amplified from the relevant genomic DNA using the following oligonucleotides (restriction endonuclease sites are bolded): Pf#1,5′-ATTTCTCGAGAATCCGAAGATAATGACG-3′ (<400>5); Pf#2,5′-ATTGCTCGAGATCGATGTTTAACATATCTTGGAATTTTTCC-3′ (<400>6); Pf#3, 5′-TTTAACTCGAGCATTTTTTAAATGAAACTG-3′ (<400>7); Pf#4,5′-CATCTAGATGTCTGAAACATCCAG-3′ (<400>8); Pc#1,5′-GGATGTTTCAGACATCTAGATGGTAAAG-3′ (<400>9); Pc#2,5′-TCACTCGAGTTAAAATAAATTAAATACAATTAATGTG-3′ (<400>10). To derive the pAMSPI and pPfM3′ fragments, Pf#1/Pf#2 and Pf#1/Pf#3 were used, respectively. to derive the pPcM3′ insert, amplicons from Pf#1/Pf#4 and Pc#1/Pc#2 were first digested with XbaI and ligated. The pPcM3′ vector is identical to pHCl except that it has a litmus 28 (NEB) backbone.

Parasite Transfection

Plasmids were transfected into P. falciparum parasites (D10 line) essentially as described (Crabb, B. V. et al. 1996 supra). After transfection and initial selection using 0.1 μM pyrimethamine for approximately 4 weeks, parasites were subjected to cycles of 1 μM pyrimethamine for 3 weeks followed by removal of the drug for 3-4 weeks. To detect homologous integration events, PCR was done on genomic DNA using a P. falciparum MSP-1 forward primer (5′-GTGAAAATAATAAGAAAGTTAACGAAGC-3′ (<400>11)) located upstream of the target sequence together with an HSP863′ reverse primer (5′-GTATATTGGGGTGATGATAAAATGAAAG-3′ (<400>12)). The identity of these products, which are only amplified if homologous integration has occurred, were confirmed by nucleotide sequencing.

Generation of P. chabaudi MSP-1₁₉ Antisera

The sequence of MSP-1₁₉ was amplified from P. chabaudi (adami D5) Dna using the oligonucleotides 5′-CACATACCCTCAATAGCTTT-3′ (<400>13) and 5′-GCTGGAAGAACTACAGAATA-3′ (<400>14), and was ligated into pFLAG (Eastman Kodak, Rochester, N.Y., USA) protein was concentrated from culture supernatants by differential ammonium sulfate precipitation, bound to a Q Sepharose ion exchange column in 25 mM histidine-HCl, pH 5.7, and eluted with a NaCl gradient on 0-0.5 M. It was then purified sing a second ion exchange chromatographic step on a Biosepra Q HyperD column again with a NaCl gradient of 0-0.5 M in a buffer of 25 mM histidine-HCl, pH 5.7. To generate αPcM19 antisera, two rabbits (A and B) were inoculated intramuscularly with 100 μg protein in Freund's complete adjuvant and were boosted twice with 100 μg protein in Freund's incomplete adjuvant. Antiserum from rabbit B was used throughout this study, except where indicated otherwise.

Growth Rate and Co-cultivation Assays

Parasites were cultured in the absence of pyrimethamine for at least 1 week before these assays. For the growth rate assay, parasites were synchronised by lysis of ‘non-ring stage’ forms with 5% (w/v) sorbital in distilled water, at 4-hour intervals, and then plated in duplicate at 0.5% parasitemia in medium containing 4% hematocrit. Thin blood smears were made every 9 hours to court parasites. Fresh media was added daily, and every 48 hours cultures were diluted 1:5 with fresh medium containing 4% hematocrit. For co-cultivation, after double synchronisation as described above, D10-PfM3′ and D10-PcM3′ ring-stage parasites were mixed at four different ratios and maintained in medium containing 4% hematocrit. Parasites were smeared at the trophozoite/schizont stage at day 1 and after two cycles at day 5. These smears were assessed by indirect immunofluorescence assay using a mixture of 4H9/19 and αPcM19 antibodies. Assays were done by incubating schizonts at 2% hematocrit for 23 hours in the presence of purified αPcM19 IgG from rabbits A and B. Assays were done in triplicate. For each well, the number of ring-stage parasites per 2,000 cells was calculated. For each slide, parasites labelled with fluorescein isothiocyanate (FITC; D10-PfM3′) and rhodamine (D10-PcM3′) were counted in about 15 individual fields that each contained at least 20 mature-stage parasites. Results are expressed as a ratio of D10-PfM3′ to D10-PcM3′ parasites.

Western Blot Analysis and Indirect Immunofluorescence

Parasite proteins were obtained from extracted enriched schizonts or merozoites preparations, and separated by 7.5% and 12% SDS-PAGE, respectively, in nonreducing conditions and transferred to PVDF membranes (Millipore, Bedford, Mass.). These were probed with either 4H9/19 antibody, diluted 1:10,000, or αPcM19 antibody, diluted 1:2,000. Parasite extracts were from parental D10, D10-PfM3′ parasites (PfM3′) and the cloned lines from D10-PcM3′ (PcM3′.1 and PcM3′.2). Molecular weight standards were obtained from BioRad (Richmond, Calif.).

For immunofluorescence, D10, D10-PfM3′ (PfM3′) and D10-PcM3′.1 (PcM3′) schizont-stage or ring-stage parasites were incubated with a mixture of 4H9/19 and αPcM19 antibodies, each diluted 1:2,000. After incubation in the presence of a mixture of FITC-conjugated antibody against mouse and rhodamine-conjugated antibody against rabbit immunoglobulins (Dako, Carpinteria, Calif.), both diluted 1:200, parasites were visualised by microscopy. The same fields were photographed with bright-field (light) and fluorescence conditions to detect the FITZ or rhodamine fluorochromes.

### EXAMPLE 3

### A New Rodent Model to Assess Blood-Stage Immunity to the Plasmodium Falciparum Antigen MSP-119 Reveals a Protective Role for Invasion Inhibitory Antibodies

Materials And Methods

Plasmids

To create the pPb-PfM19 replacement plasmid, 1.3 Kb of P. berghei MSP-1 targeting sequence was firstly fused in frame to the MSP-1₁₉ region of P. falciparum upstream from the first cysteine residue of EGF domain 1. This was achieved by PCR amplification of P. berghei ANKA and P. falciparum D10 genomic DNA (gDNA) using the oligonucleotide pairs PbF (5′-CGGGGTACCATCGATAAATACTTTACCTCTGAAGCTGTTCC (<400>15)) and PbR1 (5′-TACATGCTTAGGGTCTATACCTAATAAATC (<400>16)), and PbPfF (5′-GGTATAGACCCTAAGCATGTATGCGTAAAAAAACAATGTCCAGAA (<400>17)) and PfR (5′-TGCTCTAGATTAAATGAAACTGTATAATATTAAC (<400>18)), respectively, and sewing the products together via PCR using the primers PbF and PfR. The insertion of KpnI (underlined) and XbaI sites (boldface) into the oligonucleotides facilitated cloning of the resulting fragment into the KpnI/XbaI site of pGem4Z (Promega) and the hsp86 3′ untranslated region (UTR) from pHC2 (Crabb, B. S. (1997) supra). was cloned immediately downstream of this. The entire MSP-1/hsp86 3′ sequence, which was to serve as 5′ targeting sequence, was subsequently excised with KpnI/HindIII, the HindIII site filled in with Klenow reagent and the fragment cloned into the KpnI/lHindII site of pD_{B}D_{TmΔH}D_{B} (van Spaendonk, R. M. L., Ramesar, J., van Wigcheren, A., Eling, W., Beetsma, A. L., van Gemert, G-J., Hooghof, J., Janse, C. J. and Waters, A. P. (2001) J. Biol Chem 276:22638-22647). A 0.55 Kb 3′ targeting sequence, comprising the P. berghei MSP-1 3′ UTR, was cloned into the EcoRV/BamHI site of this vector to create pPb-PfM19. The MSP-1 3′ UTR was isolated by screening a P. berghei ANKA gDNA library (Pace, T., Birago, C., Janse, C. J., Picci, L. and Ponzi, M. 1998. Mol Biochem Parasitol 97:45-53) using the P. berghei MSP-1₁₉ sequence as a probe. This enabled the design of oligonucleotides PbM3′F (5′-GGCGATATCATAAATTATTGAAATATTTGTTGGA (<400>19)) and PbM3′R (5′-CGCGGATCCTATACAAAACATATACAAC (<400>20)), which were used to PCR amplify the P. berghei MSP-1 3′ UTR from P. berghei gDNA. The plasmid pPb-PbM19 is analogous to that of pPb-PfM19 with the exception that the entire MSP-1 5′ targeting sequence is that of P. berghei. This fragment was amplified from P. berghei ANKA gDNA using the oligonucleotides PbF and PbR2 (5′-TGCTCTAGATTAAAATATATTAAATACAAT-TAATGTG (<400>21)).

P. berghei Transfection

Eight week old Balb/c mice were used for the infection of P. berghei ANKA parasites. Infected blood at a 5% parasitemia was cultured in vitro using standard procedures (Janse, C. J., Mons, B., Croon, J. J. A. B. and van der Kaay, H. J. (1984) Int J Parasitol 14:317-320) and purified schizonts were used for the electroporation of pPb-PbM19 and pPb-PfM19 that had been linearised at the ends of the 5′ and 3′ targeting sequences using the restriction enzymes BamHI and ClaI (de Koning-Ward, T. F., Janse, C. J. and Waters, A. P. (2000). Annu Rev Microbiol 54:157-185). The resulting transfection mix was inoculated intravenously (i.v) into 2 Balb/c mice and transgenic parasites were selected using pyrimethamine (10 mg/kg bodyweight) as previously described (Menard, R., and Janse, C. J. (1997). Enzymol 13:148-159).

Nucleic Acid Analysis

Genomic DNA (gDNA) was extracted from asynchronous parasite-infected mouse blood after leukocyte removal on a CF-11 cellulose column (Whatman). PCR amplification and analysis of nucleic acids by Southern blotting was performed using standard methodologies (Sambrook, J. et al. (1989) supra).

MSP-1₁₉ GST Fusion Proteins and Generation of Antisera

The DNA sequence corresponding to the MSP-1₁₉ fragment lacking the GPI anchor sequence (amino acid Gly 1672-Ser 1766) was amplified from P. berghei ANKA gDNA using the oligonucleotides PbM19eF (5′-CGCGGATCCGGTATAGACCCTAAGCATGTATG (<400>22)) and PbM19eR (5′-GGAAGATCTTAGCTACAGAATACACCATCATAAT (<400>23)). The resulting PCR product was ligated into the BamHI site of pGEX-4T-1 and expressed as a glutathione S-transferase (GST) fusion protein (termed GST-PbM19) and rabbit antisera to GST-PbM19 was derived as described previously (O'Donnell, R. A., de Koning-Ward, T. F., Burt, R. A., Bockarie, M., Reeder, J. C., Cowman, A. F. and Crabb, B. S. (2001). J Exp Med 193:1403-1412).

Western Blot and Indirect Immunofluorescence Assay (IFA)

P. berghei-infected mouse blood was cultured in vitro to obtain cultures enriched for schizonts and merozoites. Parasites were analysed by western blot and IFA using rabbit polyconal antibodies raised against GST-PbM19 and GST-PfM19 fusion proteins and a P. falciparum MSP-1₁₉-specific monoclonal antibody 4H9/19 as described (O'Donnell, R. A. et al. (2001) supra; Cooper, J. A. et al. (1992) supra; O'Donnell, R. A., Saul, A., Cowman, A. F. and Crabb, B. S. (2000) Nat Med 6:91-95.).

Generation of Semi-immune Mice

Semi-immune Balb/c mice were generated by the administration of 1×10⁴ erythrocytes infected with either the Pb-PbM19 or Pb-PfM19 chimeric line. When the parasitemia of these mice reached approximately 5-10% they were treated for 5 consecutive days with chloroquine (CQ) (10 mg/kg bodyweight). Recrudescence was typically observed 1 week after this primary infection after which mice were administered another 5 doses of CQ. One month later mice were experimentally re-infected and then drug cured as above. Sera were obtained from individual mice 10 days after the final drug treatment to monitor MSP-1₁₉ antibodies. On the day of challenge (3 days after being bled for serology) blood smears were examined for parasites to ensure that mice were not infected with recrudescing parasites. For challenge infections, mice were injected i.p with 5×10⁶ Pb-PbM19 or Pb-PfM19 infected erythrocytes and the course of parasitemia was monitored by microscopic examination of Giemsa stained blood smears.

Serology

Antibodies reacting with recombinant P. berghei or P. falciparum MSP-1₁₉ were detected by ELISA as previously described (O'Donnell, R. A. et al. (2001) supra). Blood taken from mice prior to primary infection were used as negative controls in the ELISA. The optical density (OD) was read at 450 nm and the ELISA endpoint titres taken as the highest serum dilution that gave an OD reading 5 times above that of the control sera. Inhibition of invasion assays using the P. falciparum lines D10-PfM3′ and D10-PcMEGF were performed as described previously (O'Donnell, R. A. et al. (2001) supra).

Results

Allelic Replacement of P. berghei MSP-1₁₉ with P. falciparum MSP-1₁₉: Functional Complementation of Divergent MSP-1₁₉ Sequences

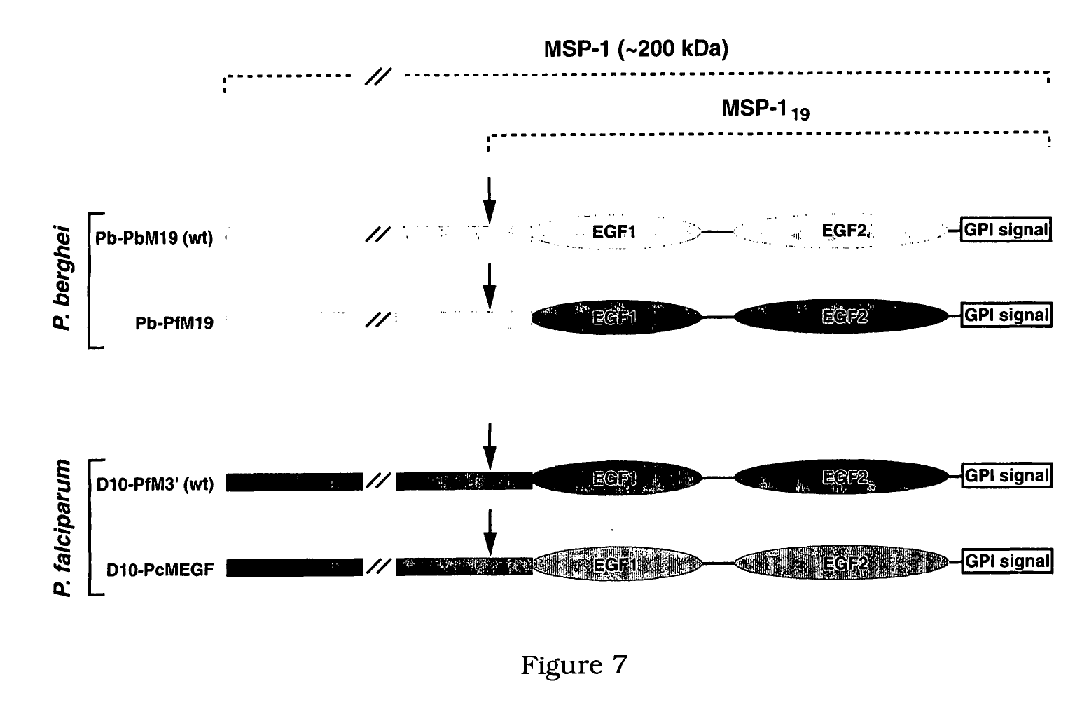

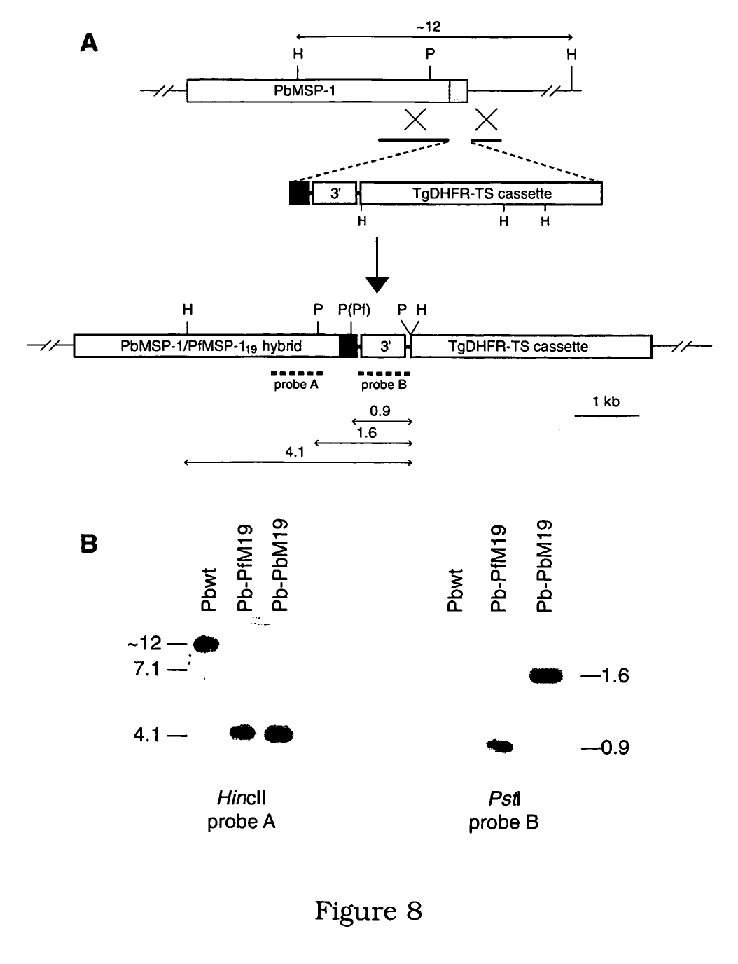

To establish whether P. falciparum MSP-1₁₉ can complement the in vivo function of the divergent P. berghei MSP-1₁₉ domain, we sought to create a P. berghei MSP-1 chimera that expresses P. falciparum MSP-1₁₉ in place of the endogenous molecule (FIG. 7). For this purpose, the transfection vector pPb-PfM19 was constructed. This plasmid was designed to integrate into the P. berghei MSP-1 locus by double-crossover homologous recombination in a manner that results in replacement of endogenous sequences encoding epidermal growth factor (EGF) domains 1 and 2, in addition to the GPI recognition sequence, with the corresponding P. falciparum (D10 line) sequence (FIGS. 7 and 8A). A second plasmid, pPb-PbM19, designed to integrate in an identical manner but resulting in a homologous MSP-1₁₉ replacement was also constructed to generate a control transfectant. Both plasmids were electroporated into the P. berghei (ANKA) line and parasites surviving 2 passages in mice under pyrimethamine selection were cloned by limiting dilution and analysed further. Southern blot analysis of gDNA showed that integration had occurred in these parasites by the expected double crossover event into MSP-1 (FIG. 8). The resulting P. berghei/P. falciparum chimeric line, which we have termed Pb-PfM19, could be distinguished from a control P. berghei transfection line, termed Pb-PbM19, by restriction endonuclease digestion with PstI (FIG. 8B). In addition, PCR amplification of gDNA using oligonucleotides specific for an integration event into MSP-1 gave the expected size products, which upon sequencing, confirmed that the expected integration event had occurred (data not shown).

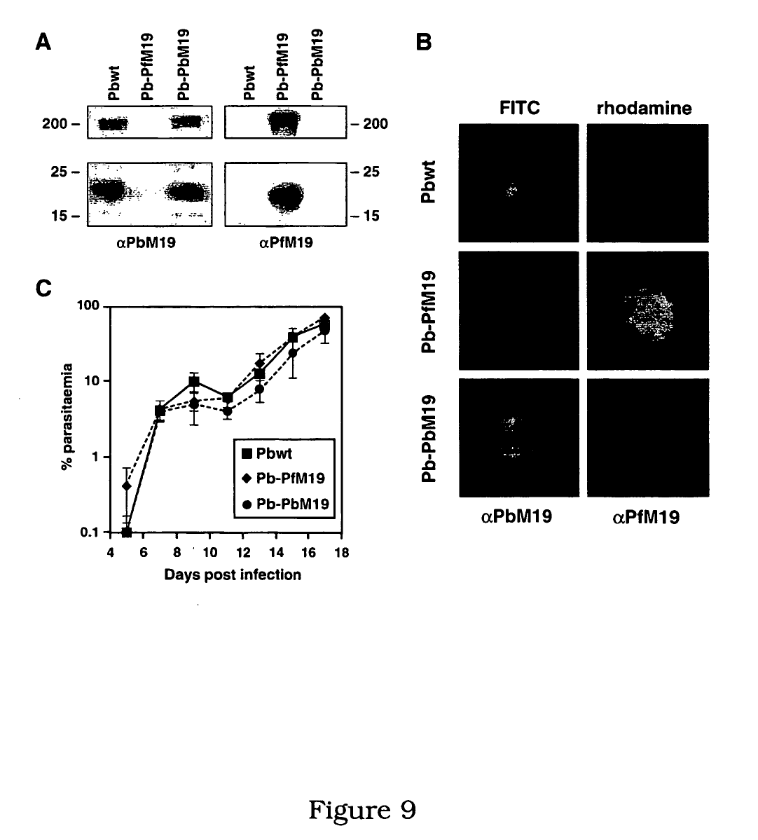

To determine whether the Pb-PfM19 and Pb-PbM19 lines expressed the expected MSP-1₁₉ domains, western blot analysis was performed on late stage parasite extracts using domain specific anti-MSP-1₁₉ antibodies (FIG. 9A). The antibodies specific for P. falciparum MSP-1₁₉ recognised both MSP-1₁₉ and an ˜200 kDa band corresponding to full-length MSP-1 in Pb-PfM19 parasites but not in Pb-PbM19 parasites. In contrast, antibodies specific for P. berghei MSP-1₁₉ only recognised MSP-1₁₉ and full-length MSP-1 in wildtype P. berghei and the transfection control line, Pb-PbM19. This demonstrates that P. falciparum MSP-1₁₉ can be correctly expressed and processed in P. berghei and that the endogenous MSP-1₁₉ gene is no longer expressed in Pb-PfM19 parasites. The localisation of MSP-1₁₉ in P. berghei lines was also assessed by double-labelling IFA. Characteristic merozoite surface labelling was observed in both chimera lines, with Pb-PfM19 parasites reacting only with the P. falciparum specific monoclonal antibody 4H9/19 while P. berghei wildtype and Pb-PbM19 chimeric parasites reacted only with rabbit anti-P. berghei MSP-1₁₉ antibodies (FIG. 9B). This confirms that the appropriate MSP-1₁₉ domain is correctly localised in both Pb-PbM19 and Pb-PfM19 parasite lines. The growth rates of the transfected lines were also examined and compared to the wildtype parasite line (FIG. 9C). All mice injected with 1×10⁴ parasites succumbed to infection over a similar time frame, regardless of which parasite line they were given. These results extend our previous finding that the function of MSP-1₁₉ during in vitro culture is conserved across divergent Plasmodium species (9, 21) to show that MSP-1₁₉ function is also conserved during the erythrocytic cycle in vivo.

A key rolefor MSP-1₁₉-specific Invasion Inhibitory Antibodies in Protection Elicited by Repeated Infection/drug Cure

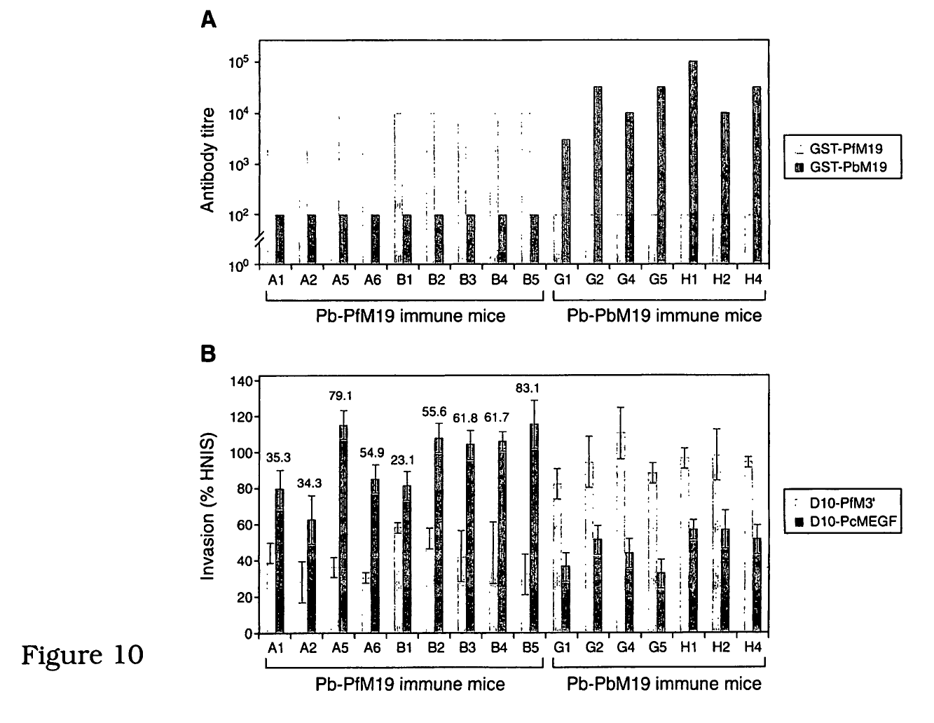

Sera from BALB/c mice that were rendered semi-immune to either Pb-PfM19 or Pb-PbM19 as a result of a low dose infection/drug cure regimen were tested for total MSP-1₁₉ antibodies in ELISA and for MSP-1₁₉ specific invasion inhibitory antibodies using a novel P. falciparum in vitro growth assay (O'Donnell, R. A. et al. (2001) supra). All mice generated a strong MSP-1₁₉ antibody response that was specific for the relevant MSP-1₁₉ domain (FIG. 10A). This data highlights the immunogenicity of this domain in the context of a low dose blood-stage infection procedure and validates the expression of the appropriate MSP-1₁₉ domains in the transfected P. berghei lines.

For the in vitro inhibition assay, the ability of a given serum to inhibit the invasion of RBC by two isogenic P. falciparum lines, D10-PfM3′ and D10-PcMEGF (FIG. 7), that differ only in their MSP-1₁₉ domains was compared. D10-PcMEGF expresses the antigenically diverse P. chabaudi MSP-1₁₉ polypeptide and so is not recognised by P. falciparum MSP-1₁₉ specific antibodies. Hence, P. falciparum MSP-1₁₉ specific invasion inhibitory activity of a given serum can be calculated by determining the difference in invasion rates of D10-PfM3′, which utilises the wt P. falciparum MSP-1₁₉ domain for invasion, and D10-PcMEGF in the presence of the test serum. All sera from Pb-PfM19 mice inhibited D10-PfM3′ parasites far more effectively than D10-PcMEGF parasites (FIG. 10B). Conversely, all sera from Pb-PbM19 immune mice inhibited D10-PcMEGF more effectively than wt P. falciparum. Since P. chabaudi and P. berghei are closely related rodent parasites with somewhat conserved MSP-1₁₉ domains (73% identity) a degree of antigenic cross-reactivity was expected here. However, many epitopes differ between the MSP-1₁₉ domains of rodent malaria parasites (Benjamin, P. A., Ling, I. T., Clottey, G., Spencer, Valero, L. M., Ogun, S. A., Fleck, S. L., Walliker, D., Morgan, W. D., Birdsall, B., Feeney, J. and Holder, A. A. (1999). Mol Biochem Parasitol 104:147-156), hence the invasion-inhibitory activity of these sera cannot be accurately determined.

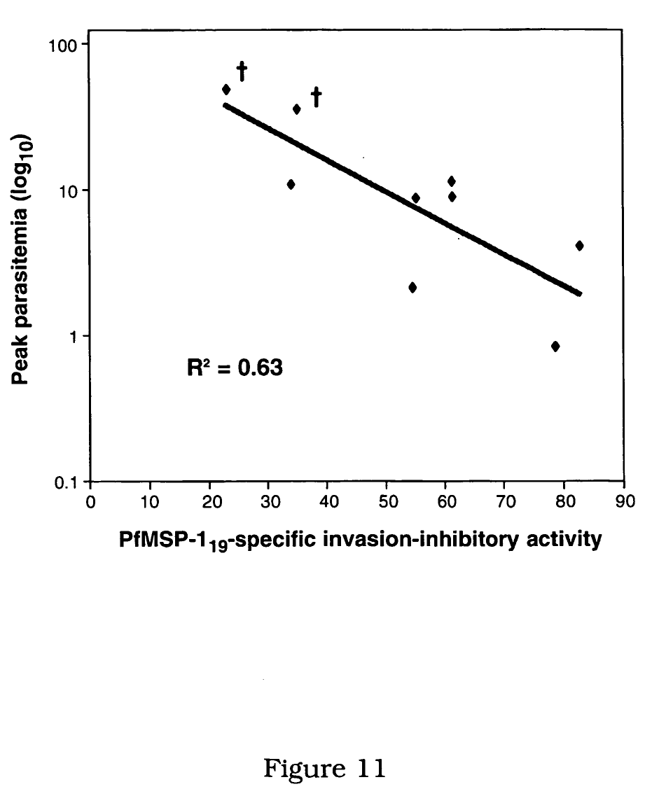

To determine if there is an association between the levels of MSP-1₁₉ specific invasion inhibitory antibodies present in mouse serum and degree of protection from a subsequent parasite challenge, Pb-PfM19 semi-immune mice were administered a high dose (5×10⁶) of Pb-PfM19 infected erythrocytes 3 days after they had been bled for the serological analyses described above. Following challenge, the course of parasitemia was determined and plotted against levels of P. falciparum MSP-1₁₉-specific invasion inhibitory antibodies (FIG. 11). Strong evidence of regression was observed (R^{2=0.63}; P=0.01 by Anova), implicating a substantial role for MSP-1₁₉ specific inhibitory antibodies in controlling a blood-stage infection. A similarly significant regression curve was evident when invasion inhibition was plotted against cumulative parasitemia in these mice (R²=0.56; P=0.02). Also, a 2-sided Rank Correlation Test, a more stringent analysis that does not assume a linear relationship between 2 parameters, also demonstrated significance for peak parasitemia versus MSP-1₁₉-specific invasion inhibition (P=0.05). Importantly, all mice had very similar anti-PfMSP-1₁₉ ELISA antibody endpoint titres (FIG. 10A), hence, no association between IgG levels and protection were observed.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Benjamin, P. A., Ling, I. T., Clottey, G., Spencer, Valero, L. M., Ogun, S. A., Fleck, S. L., Walliker, D., Morgan, W. D., Birdsall, B., Feeney, J. and Holder, A. A. (1999) Mol Biochem Parasitol 104:147-156.
Blackman, M. J., Heidrich, H.-G., Donachie, S., McBridge, J. S. and Holder, A. A. (1990) J. Exp. Med 172:379-382.
Blackman, M. J., Scott Finnigan, T. J., Shai, S. and Holder, A. A. (1994) J. Exp. Med. 180:389-393.
Bouharoun-Tayoun, H., Attanath, P., Sabchareon, A., Chongsuphajaisiddhi, T. and Druilhe, P. (1990) J. Exp. Med 172:1633-1641.
Brown, G. V., Anders, R. F., Mitchell, G. F. and Heywood, P. F. (1982) Nature 297:591-593.
Brown, G. V., Anders, R. F. and Knowles, G. (1983) Infect. Immun. 39:1228-1235.
Cattani, J. A., Tulloch, J. L., Vrbova, H., Jolley, D., Gibson, F. D., Moir, J. S., Heywood, P. F., Alpers, M. P., Stevenson, A. and Clancy, R. (1986) Am. J. Trop. Med Hyg. 35:3-15.
Chang, S. P., Gibson, H. L., Lee Ng, C. T., Bar, P. J. and Hui, G. S. (1992) J. Immunol. 149:548-555.
Chappel, J. A. and Holder, A. A. (1993) Mol. Biochem. Parasitol. 60:303-311.
Cooper, J. A., Cooper, L. T. and Saul, A. J. (1992) Mol. Biochem. Parasitol. 51:301-312.
Coppel, R. L. and Biano, A. E., Culvenor, J. G., Crewther, P. E., Brown, G. V., Anders, R. F. and Kemp, D. J. (1987) Mol. Biochem. Parasitol. 25:73-81.
Crabb, B. S. and Cowman, A. F. (1996) Proc. Natl. Acad. Sci. USA. 93:7289-7294.
Crabb, B. S., Triglia, T., Waterkeyn, J. F. and cowman, A. F. (1997) Mol. Biochem. Parasitol. 90:131-144.
de Koning-Ward, T. F., Janse, C. J. and Waters, A. P. (2000). Annu Rev Microbiol 54:157-185.
Digs, C. L., Ballou, W. R. and Miller, L. H. (1993) Parasitol. Today. 9:300-302.
Egan, A., Burghaus, P., Druilhe, P., Holder, A. and Riley, E. (1999) Parasite Immunol. 21:133-139.
Egan, A. F., Chappel, J. A., Burghaus, P. A., Morris, J. S., McBride, J. S., Holder, A. A., Kaslow, D. C. and Riley, E. M. (1995) Infect. Immun. 63:456-466.
Fidock, D. A. and Wellems, T. E. (1997) Proc. Natl. Acad. Sci. USA. 94:10931-10936.
Good, M. F., Kaslow, D. C. and Miller, L. H. (1998) Annu. Rev. Immunol. 16:57-87.
Hodder, A. N., Crewther, P. E. and Anders, R. F. (2001) Infect Immun. 69:3286-3294.
Janse, C. J., Mons, B., Croon, J. J. A. B. and van der Kaay, H. J. (1984) Int J Parasitol 14:317-320.
Lambros, C. and Vanderberg, J. P. (1979) J Parasitol. 65:418-420.
Marsh, K., Otoo, L., Hayes, R. J., Carson, D. C. and Greenwood, B. M. (1989) Trans. R. Soc. Trop. Med. Hyg. 83:293-303.
McGregor, I. A. and Wilson, R. M. J. (1988) Specific immunity acquired in man. In Malaria, Principles and Practices of Malariology. W. H. Wernsdorfer and I. A. McGregor, editors. Churchill Livingston, Inc., New York. 559-619.
Menard, R., and Janse, C. J. (1997). Enzymol 13:148-159
Miller, L. H., Roberts, T., Shahabuddin, M. and McCutchan, T. F. (1993) Mol. Biochem. Parasitol. 59:1-14.
Mohan, K. and Stevenson, M. M. (1998) Acquired immunity to asexual blood stages. In Malaria, parasite Biology, Pathogenesis and Protection. I. W. Sherman, editor. ASM Press, Washington, D.C. 467-493.
O'Donnell, R. A., Saul, A., Cowman, A. F. and Crabb, B. S. (2000) Nat. Med. 6:91-95.
O'Donnell, R. A., de Koning-Ward, T. F., Burt, R. A., Bockarie, M., Reeder, J. C., Cowman, A. F. and Crabb, B. S. (2001). J Exp Med 193:1403-1412.

Pace, T., Birago, C., Janse, C. J., Picci, L. and Ponzi, M. (1998) Mol Biochem Parasitol 97:45-53
Sambrook, J., Fritsch, E. F. and Maniatis, T. (1989) Molecular Cloning: A Laboratory Manual. Cold Spring Harbor Laboratory Press, New York.
Smith, D. B. and Johnson, K. S. (1988) Gene 67:31-40.
Tanabe, K., Mackay, M., Goman, M. and Scaife, J. G. (1987) J. Mol. Biol. 195:273-287.
Triglia, T., Healer, J., Caruana, S. R., Hodder, A. N., Anders, R. F., Crabb, B. S. and Cowman, A. F. (2000) Mol. Microbiol. 38:706-718.

van Spaendonk, R. M. L., Ramesar, J., van Wigcheren, A., Eling, W., Beetsma, A. L., van Gemert, G.-J., Hooghof, J., Janse, C. J. and Waters, A. P. (2001) J Biol Chem. 276:22638

TABLE I
Reactivity in ELISA of Sera from Malaria-infected Humans and
Mice Against Recombinant P. falciparum and P. chabaudi MSP-1₁₉

Mean OD_{450 nm}*

| Sera | n | GST-PfM19 | GST-PcM19 |
|---|---|---|---|
| PNG-B | 47 | 0.846 ± 0.200 | 0.072 ± 0.158 |
| PNG-M | 33 | 0.481 ± 0.374 | 0.022 ± 0.032 |
| Pc immune | 6 | 0.016 ± 0.012 | 0.613 ± 0.197 |

*Mean OD readings after subtraction of GST reactivity ± SD. All sera were tested at a 1:3,200 dilution.
‡ Number of sera above an OD value which equalled the mean plus three SDs of that registered in replicate assays with pooled normal human or mouse sera where relevant.
The cut-off values ranged from 0.078 to 0.180 depending on the serum/antigen combination.

1. A method of detecting the presence of a functionally modulatory immunointeractive molecule in a biological sample, which immunointeractive molecule is directed to a pathogen derived antigen, said method comprising:
(i) contacting a pathogen expressing an epitopically distinct form of said antigen with said sample for a time and under conditions sufficient to facilitate immunointeraction;
(ii) contacting a pathogen expressing an epitopically native form of said antigen with said sample for a time and under conditions sufficient to facilitate immunointeraction;
(iii) assessing the level of functional activity of the pathogens of step (i) and step (ii)
wherein modulation in the functional activity of the pathogen of step (ii) relative to the pathogen of step (i) is indicative of the presence of a functionally inhibitory immunointeractive molecule in said sample.

2. The method according to claim 1 wherein said modulation is down-regulation.

3. The method according to claim 1 or 2 wherein said immunointeractive molecule is an antibody.

4. The method according to claim 1 or 2 or 3 wherein said method is performed in vitro.

5. The method according to claim 4 wherein said pathogen is a parasite.

6. The method according to claim 1 or 2 or 3 wherein said method is performed in vivo.

7. The method according to claim 6 wherein said pathogen is a parisite.

8. The method according to claim 5 or 7 wherein said parasite is a Plasmodium species.

9. The method according to claim 8 wherein the Plasmodium species to which said antibody is directed is one of Plasmodium falciparum, Plasmodium malariae, Plasmodium ovare or Plasmodium vivax.

10. The method according to claim 9 wherein said antigen is any domain of MSP-1.

11. The method according to claim 10 wherein said domain of MSP-1 is the block 2 N-terminal domain or the block 17 C-terminal domain.

12. The method according to claim 9 wherein said antigen is the apical membrane domain (AMA-1).

13. The method according to claim 9 wherein said antigen is the merozoite surface protein 2, 3, 4 or 5 (MSP-2, MSP-3, MSP-4 or MSP-5).

14. The method according to claim 9 wherein said antigen is the rhoptry associated protein 2 (RAP-2).

15. The method according to claim 9 wherein said antigen is the erythrocyte binding antigen (EBA-175).

16. The method according to claim 9 wherein said antigen is the circumsprozoite antigen (CSP).

17. The method according to claim 4 or 6 wherein said immunointeractive molecule is directed to a Plasmodium chabaudi antigen, said pathogen of step (i) is wild-type Plasmodium falciparum and said pathogen of step (ii) is Plasmodium falciparum expressing said Plasmodium chabaudi antigen.

18. The method according to claim 17 wherein said antigen is MSP-1₁₉, said Plasmodium falciparum pathogen of step (i) is the strain D10 and said Plasmodium falciparum pathogen of step (ii) is the strain D10-P MEGF or D10-PcM3′.

19. The method according to claim 6 wherein said method is performed in vivo in mice, said immunointeractive molecule is directed to a Plasmodium falciparum antigen, said pathogen of step (ii) is wild-type Plasmodium berghei and said pathogen of step (ii) is Plasmodium berghei expressing said Plasmodium falciparum antigen.

20. The method according to claim 19 wherein said antigen is MSP-1₁₉ and said Plasmodium berghei of step (ii) is the strain Pb-PfM19.

21. An isolated malaria pathogen expressing a non-wild-type form of one or more antigens derived from said pathogen.

22. The malaria pathogen of claim 21 wherein said pathogen is P. falciparum.

23. The Plasmodium falciparum of claim 22 wherein said antigen is MSP-1₁₉.

24. The Plasmodium falciparum of claim 23 wherein said MSP-1₁₉ antigen corresponds to all or part of the Plasmodium chabaudi MSP-1₁₉ region.

25. The Plasmodium falciparum according to claim 24 corresponding to strain D10-PcMEGF.

26. The Plasmodium falciparum according to claim 24 corresponding to strain D10-PcM3′.

27. The malaria pathogen of claim 21 wherein said pathogen is Plasmodium berghei.

28. The Plasmodium berghei of claim 27 wherein said antigen is MSP-1₁₉.

29. The Plasmodium berghei of claim 28 wherein said antigen corresponds to all or part of the Plasmodium falciparum MSP-1₁₉ region.

30. The Plasmodium berghei according to claim 29 corresponding to strain Pb-PfM19.

31. A method of assessing any one or more qualitative and/or quantitative aspects of an immune response directed to a pathogen in accordance with the method of any one of claims 1-20.


SEQUENCE LISTING (25 sequences)

SEQ ID NO: 1 - DNA, 28 bp, Artificial Sequence
  Oligonucleotide primer
atttctcgagaatccgaagataatgacg

SEQ ID NO: 2 - DNA, 54 bp, Artificial Sequence
  Oligonucleotide primer
gaaacatccagcattttctggaagtttgttcctatgcattggtgttgtgaaatg

SEQ ID NO: 3 - DNA, 31 bp, Artificial Sequence
  Oligonucleotide primer
cgcggatccaacatttcacaacaccaatgcg

SEQ ID NO: 4 - DNA, 35 bp, Artificial Sequence
  Oligonucleotide primer
ggaagatcttaactgcagaaaataccatcgaaaag

SEQ ID NO: 5 - DNA, 28 bp, Artificial Sequence
  Oligonucleotide primer
atttctcgagaatccgaagataatgacg

SEQ ID NO: 6 - DNA, 41 bp, Artificial Sequence
  Oligonucleotide primer
attgctcgagatcgatgtttaacatatcttggaatttttcc

SEQ ID NO: 7 - DNA, 30 bp, Artificial Sequence
  Oligonucleotide primer
tttaactcgagcattttttaaatgaaactg

SEQ ID NO: 8 - DNA, 24 bp, Artificial Sequence
  Oligonucleotide primer
catctagatgtctgaaacatccag

SEQ ID NO: 9 - DNA, 28 bp, Artificial Sequence
  Oligonucleotide primer
ggatgtttcagacatctagatggtaaag

SEQ ID NO: 10 - DNA, 37 bp, Artificial Sequence
  Oligonucleotide primer
tcactcgagttaaaataaattaaatacaattaatgtg

SEQ ID NO: 11 - DNA, 28 bp, Artificial Sequence
  Oligonucleotide primer
gtgaaaataataagaaagttaacgaagc

SEQ ID NO: 12 - DNA, 28 bp, Artificial Sequence
  Oligonucleotide primer
gtatattggggtgatgataaaatgaaag

SEQ ID NO: 13 - DNA, 20 bp, Artificial Sequence
  Oligonucleotide primer
cacataccctcaatagcttt

SEQ ID NO: 14 - DNA, 20 bp, Artificial Sequence
  Oligonucleotide primer
gctggaagaactacagaata

SEQ ID NO: 15 - DNA, 41 bp, Artificial Sequence
  Oligonucleotide primer
cggggtaccatcgataaatactttacctctgaagctgttcc

SEQ ID NO: 16 - DNA, 30 bp, Artificial Sequence
  Oligonucleotide primer
tacatgcttagggtctatacctaataaatc

SEQ ID NO: 17 - DNA, 45 bp, Artificial Sequence
  Oligonucleotide primer
ggtatagaccctaagcatgtatgcgtaaaaaaacaatgtccagaa

SEQ ID NO: 18 - DNA, 34 bp, Artificial Sequence
  Oligonucleotide primer
tgctctagattaaatgaaactgtataatattaac

SEQ ID NO: 19 - DNA, 34 bp, Artificial Sequence
  Oligonucleotide primer
ggcgatatcataaattattgaaatatttgttgga

SEQ ID NO: 20 - DNA, 28 bp, Artificial Sequence
  Oligonucleotide primer
cgcggatcctatacaaaacatatacaac

SEQ ID NO: 21 - DNA, 37 bp, Artificial Sequence
  Oligonucleotide primer
tgctctagattaaaatatattaaatacaattaatgtg

SEQ ID NO: 22 - DNA, 32 bp, Artificial Sequence
  Oligonucleotide primer
cgcggatccggtatagaccctaagcatgtatg

SEQ ID NO: 23 - DNA, 34 bp, Artificial Sequence
  Oligonucleotide primer
ggaagatcttagctacagaatacaccatcataat

SEQ ID NO: 24 - protein, 117 aa, plasmodium falciparum
DMLNISQHQCVKKQCPENSGCFRHLDEREECKCLLNYKQEGDKCVENPNPTCNENNGGCDADATCTEEDSGSSRKKITCECTKPDSYPLFDGIFCSSSNFLGISFLLILMLILYSFI

SEQ ID NO: 25 - protein, 122 aa, plasmodium falciparum
DLLGIGSNHVCIGTNIPENAGCFRYDDGKEEWRCLLGFKKNDDNTRCEKDTGATCSNNNGGCDPSADCREVENADRKPSKKIVCTCKEPTPNAYYDGVFCSSSGFMGLSILLIITLIVFNLF